(19) United States
(12) Patent Application Publication
(10) Pub. No.: US 2016/0009694 A1
(43) Pub. Date: Jan. 14, 2016

(54) CYCLOHEXANEDIAMINE COMPOUNDS AND METHODS FOR THEIR PREPARATION

(71) Applicant: PORTOLA PHARMACEUTICALS, INC., South San Francisco, CA (US)

(72) Inventor: ANJALI PANDEY, Fremont, CA (US)

(21) Appl. No.: 14/771,768

(22) PCT Filed: Mar. 14, 2014
(86) PCT No.: PCT/US2014/027711

§ 371 Date: Aug. 31, 2015

Related U.S. Application Data
(60) Provisional application No. 61/792,318, filed on Mar. 15, 2013.

(51) Int. Cl. C07D 403/12; C07D 239/48; C07C 213/02; C07C 303/28; C07C 269/06; C07C 269/04

(52) U.S. Cl. CPC C07D 403/12; C07C 269/06; C07C 269/04; C07C 213/02; C07C 303/28; C07D 239/48; C07C 2101/14

(57) ABSTRACT

The present invention provides processes for the preparation of cyclohexanediamine compounds of formula Ia and intermediates thereof. The compounds are useful as Syk kinase inhibitors and in various pharmaceutical compositions, and particularly useful for treating conditions mediated at least in part by Syk kinase activity.

### CROSS REFERENCE TO RELATED APPLICATIONS

This application claims the benefit of U.S. Provisional Patent Application No. 61/792,318, filed Mar. 15, 2013; the entire disclosure of which is incorporated herein by reference.

### FIELD OF THE INVENTION

The present disclosure relates to methods for preparing inhibitors of Spleen tyrosine kinase (Syk) and intermediates thereof.

### BACKGROUND OF THE INVENTION

Spleen tyrosine kinase (Syk) plays an important role in a number of pathologies including cardiovascular, inflammatory, and autoimmune diseases, and consequently is an important target in the development of inhibitors for treating these diseases. Substituted pyrimidinediamine compounds have been found to be potent inhibitors of Syk. Examples of such inhibitors include cyclohexyldiamine compounds 4-(3-(1H-1,2,3-triazol-2-yl)phenylamino)-2-((1R,2S)-2-aminocyclohexylamino)pyrimidine-5-carboxamide and 2-((1R,2S)-2-aminocyclohexylamino)-4-(3-(pyrimidin-2-yl)phenylamino)pyrimidine-5-carboxamide disclosed in WO 2009/136995. However, a need exists for improved methods for preparing cyclohexyldiamine substituted pyrimidinediamine Syk inhibitors and their salts. The present invention fulfills the above needs by providing more efficient and cost-effective processes and intermediates for making these compounds.

### BRIEF SUMMARY OF THE INVENTION

The present invention, provides in one aspect, processes for preparing cyclohexylamine containing compounds having activity as Syk inhibitors. In one aspect, provided is a process for preparing a compound of Formula (Ia):

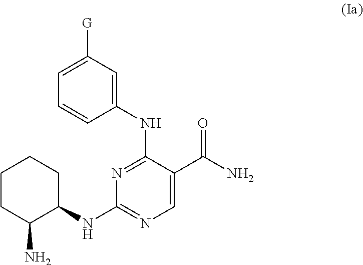

or a salt or tautomer thereof wherein G is heteroaryl;

the process comprising contacting a compound of Formula (Ib) with formamide and a C₁₋₈alkoxide to form a compound of Formula (Ic):

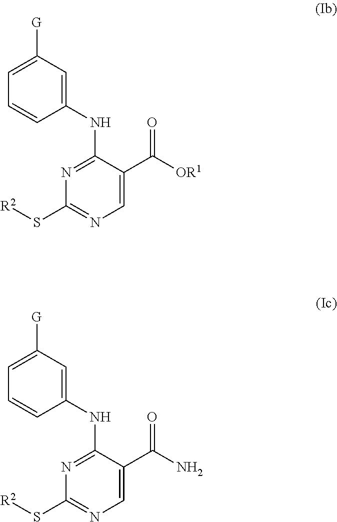

wherein R¹ and R² are independently C₁-C₈alkyl; and converting a compound of Formula (Ic) to a compound of Formula (Ia). In other aspects, provided are synthetic intermediates used in the preparation of these compounds and methods for their preparation.

In another aspect, provided is a process for preparing a compound of Formula (II):

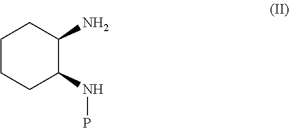

the process comprising:

(a) contacting racemic mixture (IV) with D-mandelic acid

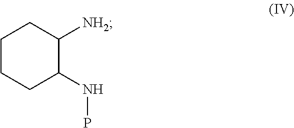

(b) isolating D-mandelic acid salt (V):

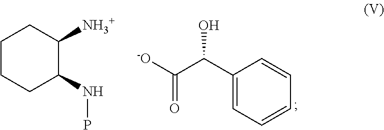

and

(c) contacting the D-mandelic acid salt (V) with base to provide the compound of Formula (II).

In another aspect, provided is a process for preparing a compound of Formula (IIa):

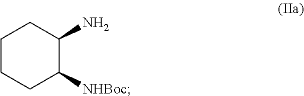 [SMILES: CC(C)(C)OC(=O)N[C@H]1CCCC[C@H]1N]

wherein Boc is —C(O)OC(CH₃)₃, the process comprising:

(a) contacting cyclohexene oxide with benzylamine to provide a racemic mixture of a compound of Formula (VI) wherein R¹ is —CH₂Ph:

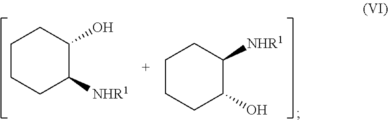

(b) contacting the racemic mixture (VI) with D-mandelic acid;

(c) isolating D-mandelic acid salt (VII) wherein R¹ is —CH₂Ph:

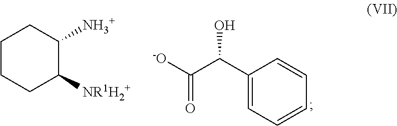

(d) contacting salt (VII) with base to provide a compound of Formula (VIII) wherein Y is OH and R¹ is —CH₂Ph:

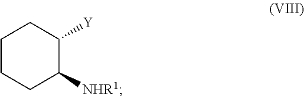

(e) contacting a compound of Formula (VIII) wherein Y is OH and R¹ is —CH₂Ph with a reducing agent and then with di-tert-butyl dicarbonate to provide a compound of Formula (VIII) wherein Y is OH and R¹ is —C(O)OC(CH₃)₃;

(f) contacting a compound of Formula (VIII) wherein Y is OH and R¹ is —C(O)OC(CH₃)₃ with an alkylsulfonylhalide to form a compound of Formula (VIII) wherein Y is —OS(O)₂alkyl and R¹ is —C(O)OC(CH₃)₃;

(g) contacting a compound of Formula (VIII) wherein Y is —OS(O)₂C₁₋₈alkyl and R¹ is —C(O)OC(CH₃)₃ with MN₃ where M is selected from the group consisting of Li, K, or Na to form a compound of Formula (IX) wherein R¹ is —C(O)OC(CH₃)₃:

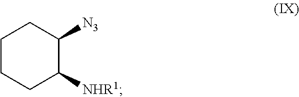

and

(h) contacting a compound of Formula (IX) wherein R¹ is —C(O)OC(CH₃)₃ with a reducing agent to provide the compound of Formula (IIa).

These and other aspects of the invention are further described in the description that follows.

### DETAILED DESCRIPTION OF THE INVENTION

1. Abbreviations and Definitions

As used herein, the below terms have the following meanings unless specified otherwise:

The abbreviations used herein are conventional, unless otherwise defined. The following abbreviations are used: AcOH=acetic acid, aq.=aqueous, atm=atmosphere, Boc=t-butoxycarbonyl, Bn=benzyl, ° C.=degrees celcius, conc=concentrated, mCPBA=m-chloroperoxybenzoic acid (3-chloroperbenzoic acid), DCM=dichloromethane, DMF=dimethyl formamide, DMSO=dimethyl sulfoxide, Et₃N=triethylamine, EtOAc=ethyl acetate, ee=enantiomeric excess, eq=equivalents, g=gram, formamide=HC(O)NH₂, GC=gas chromatography, H₂=hydrogen; HPLC=high pressure liquid chromatography, LC=liquid chromatography, h=hour, IPA=isopropanol, kg=kilogram, MTBE=methyl tert-butyl ether, mmol=millimole, mL=milliliter, M=molar, N=Normal, NMP=N-methylpyrrolidone, NMR=nuclear magnetic resonance, Pd/C=palladium on carbon, ppm=parts per million, psi=pound per square inch, rp=reverse phase, sat=saturated, RT=room temperature, TEA=triethylamine, and TLC=thin layer chromatography.

It is noted here that as used in this specification and the appended claims, the singular forms “a,” “an,” and “the” include plural reference unless the context clearly dictates otherwise.

“Alkyl,” by itself or as part of another substituent, means, unless otherwise stated, a straight or branched chain, fully saturated aliphatic hydrocarbon radical having the number of carbon atoms designated. For example, “C₁₋₈alkyl” refers to a hydrocarbon radical straight or branched, containing from 1 to 8 carbon atoms that is derived by the removal of one hydrogen atom from a single carbon atom of a parent alkane. Alkyl includes branched chain isomers of straight chain alkyl groups such as isopropyl, t-butyl, isobutyl, sec-butyl, and the like. Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms.

“Heteroaryl” refers to a cyclic or polycyclic aromatic radical that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom or through a carbon atom and can contain 5 to 10 carbon atoms. Non-limiting examples of heteroaryl groups include 1-pyrrolyl, 2-pyrrolyl, 3-pyrrolyl, 1-pyrazolyl, 3-pyrazolyl, 2-imidazolyl, 4-imidazoyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, and 1H-1,2,3-triazol-2-yl.

“Tautomer” refers to alternate forms of a molecule that differ in the position of a proton, such as the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

The term “salts” is meant to include salts prepared with relatively nontoxic acids. Acid addition salts can be obtained by contacting the neutral form of the compound for Formula (Ia) or intermediates disclosed herein with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M. et al., “Pharmaceutical Salts,” Journal of Pharmaceutical Science, 66:1-19, 1977). Certain specific compounds of the present invention contain both basic and acidic functionalities that allow the compounds to be converted into either base or acid addition salts.

The neutral forms of the compound for Formula (Ia) may be regenerated by contacting the salt with a base or acid and isolating the parent compound in the conventional manner. [0016] As used herein, the term “Syk” refers to a spleen tyrosine kinase (RefSeq Accession No. P-043405) or a variant thereof that is capable of mediating a cellular response to T-cell receptors in vitro or in vivo. Syk variants include proteins substantially homologous to native Syk, i.e., proteins having one or more naturally or non-naturally occurring amino acid deletions, insertions or substitutions (e.g., Syk derivatives, homologs and fragments). The amino acid sequence of Syk variant preferably is at least about 80% identical to a native Syk, more preferably at least about 90% identical, and most preferably at least about 95% identical.

The term “Syk inhibitor” refers to any agent that inhibits the catalytic activity of spleen tyrosine kinase.

2. Compounds of the Invention

In one group of embodiments, the compound has Formula (Ia):

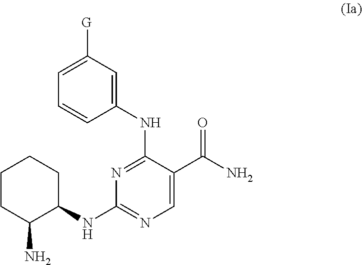

or a salt or tautomer thereof wherein G is heteroaryl.

3. Methods of Preparation of Compounds of the Invention and Synthetic Intermediates

In one group of embodiments, provided is a process for preparing a compound of Formula (Ia):

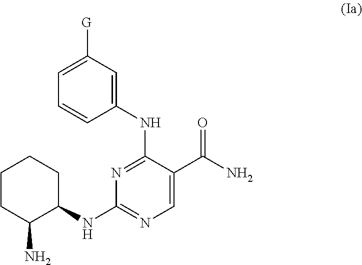

or a tautomer, salt, or hydrate thereof wherein G is heteroaryl;
the process comprising (a) contacting a compound of Formula (Ib) with a compound of Formula (II) to form a compound of Formula (Ic):

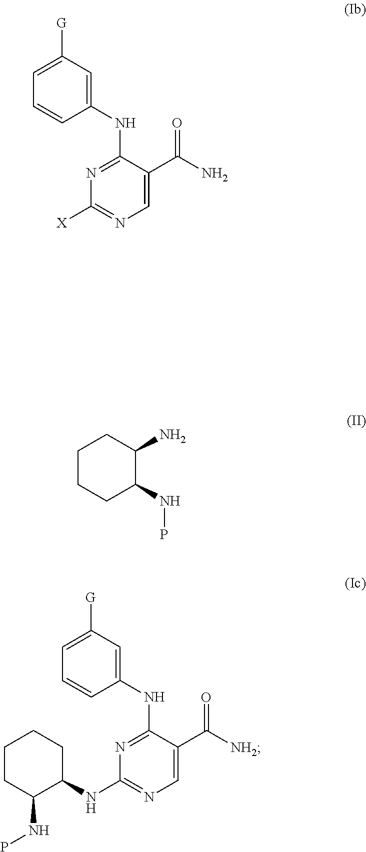

wherein X is a leaving group; and P is a protecting group;
and (b) further converting a compound of Formula (Ic) to a compound of Formula (Ia).

Methods for the conversion of (Ic) to (Ia) include those disclosed herein and in WO 2009/136995. Suitable protecting groups include, but are not limited to, t-butoxycarbonyl (Boc), allyloxycarbonyl (Alloc), benzyloxycarbonyl (Cbz), trifluoroacetyl, phthalimido, benzyl, triphenylmethyl (trityl), and benzylidene. Protecting groups can be removed using one or more deprotecting reagents including, but not limited to, hydrochloric acid, acetic acid, trifluoroacetic acid, tosylic acid, sulfuric acid, trimethylsilyl iodide, trimethylsilyl chloride, trimethylsilyl triflate, tetrakis(triphenylphosphine)palladium (0), tributyltin hydride, phenylsilane, palladium on carbon with hydrogen gas, sodium borohydride, hydrazine, and phenylhydrazine. Other suitable protecting groups and deprotecting reagents are known to those of skill in the art as described, for example, by Wuts & Green (Protective Groups in Organic Synthesis, 4^{th} Ed. Hoboken: Wiley-Interscience, 2007).

In one group of embodiments, step (b) further comprises (i) contacting a compound of Formula (Ic) with a deprotecting reagent and subsequently with a base to provide a compound of Formula (Ia) as a free base; and

(ii) optionally contacting the free base of Formula (Ia) with an acid to provide a salt of Formula (Ia).

In one group of embodiments, the process further comprises contacting a compound of Formula (Id) with a formamide and an C₁₋₈alkoxide to form a compound of Formula (Ib):

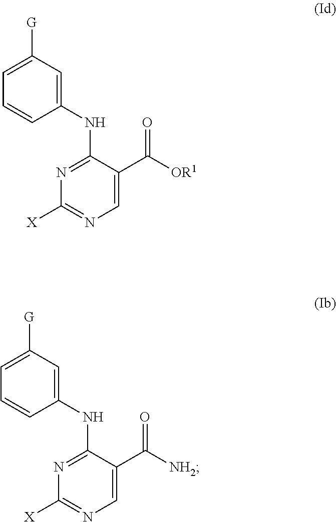

wherein R¹ is is C₁-C₈alkyl.

In one group of embodiments, X is halo or —S(O)ₙC₁-C₈alkyl. In one group of embodiments, P is t-Boc.

In one group of embodiments, the alkoxide in the conversion of (Ic) to (Id) is sodium ethoxide. In one group of embodiments, in part (a) X is —SCH3 which is contacted with an oxidizing agent before the compound of formula (Ib) is contacted with a compound of formula (II). In one group of embodiments, the oxidizing agent is 3-chloroperbenzoic acid. In one group of embodiments, the deprotecting reagent of part (b) is hydrochloric acid. In one group of embodiments, the acid of part (b) is acetic acid and the compound of Formula (Ia) is an acetate salt.

In one group of embodiments, the compound of Formula (II) is prepared by:
(a) contacting racemic mixture (IV) with D-mandelic acid

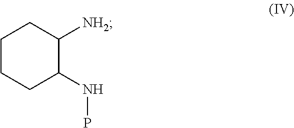

(b) isolating D-mandelic acid salt (V):

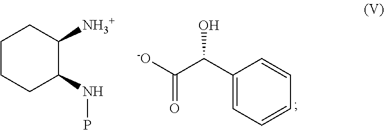

and
(c) contacting the D-mandelic acid salt (V) with base to provide the compound of Formula (II).

In one group of embodiments, the compound of Formula (II) has an enantiomeric excess of at least 98% e.e. In one group of embodiments, the base is potassium carbonate.

In one group of embodiments, the compound of Formula (III) is prepared by
(a) contacting 1,2-cyclohexanediamine with DL-tartaric acid to provide a tartaric acid salt of trans-1,2-cyclohexanediamine; and
(b) removing cis-1,2-cyclohexanediamine from the tartaric acid salt of trans-1,2-cyclohexanediamine and isolating cis-1,2-cyclohexanediamine (III).

In one group of embodiments, the compound of Formula (IV) is prepared by:
(a) contacting 1,2-cyclohexanediamine with DL-tartaric acid to provide a tartaric acid salt of trans-1,2-cyclohexanediamine; and
(b) removing cis-1,2-cyclohexanediamine from the tartaric acid salt of trans-1,2-cyclohexanediamine and isolating cis-1,2-cyclohexanediamine (III):

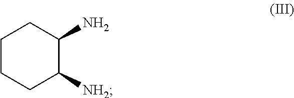

and
(c) contacting cis-1,2-cyclohexanediamine (III) with an acid and di-tert-butyl dicarbonate to provide a racemic mixture of the compound of Formula (IV).

In one group of embodiments, the compound of Formula (II) is prepared by:
(a) contacting cyclohexene oxide with benzylamine to provide a racemic mixture of a compound of Formula (VI) wherein R¹ is —CH₂Ph:

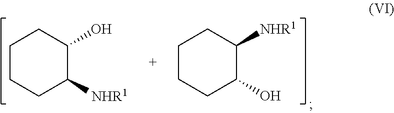

(b) contacting the racemic mixture (VI) with D-mandelic acid;
(c) isolating D-mandelic acid salt (VII) wherein R¹ is —CH₂Ph:

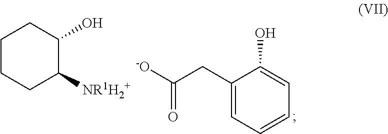

(d) contacting salt (VII) with base to provide a compound of Formula (VIII) wherein Y is OH and R¹ is —CH₂Ph:

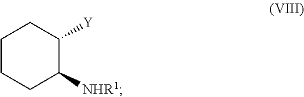

(e) contacting a compound of Formula (VIII) wherein Y is OH and R¹ is —CH₂Ph with a reducing agent and then with di-tert-butyl dicarbonate to provide a compound of Formula (VIII) wherein Y is OH and R¹ is —C(O)OC(CH₃)₃;
(f) contacting a compound of Formula (VIII) wherein Y is OH and R¹ is —C(O)OC(CH₃)₃ with an alkylsulfonylhalide to form a compound of Formula (VIII) wherein Y is —OS(O)₂alkyl and R¹ is —C(O)OC(CH₃)₃;
(g) contacting a compound of Formula (VIII) wherein Y is —OS(O)₂C₁₋₈alkyl and R¹ is —C(O)OC(CH₃)₃ with MN₃ where M is selected from the group consisting of Li, K, or Na to form a compound of Formula (IX) wherein R¹ is —C(O)OC(CH₃)₃:

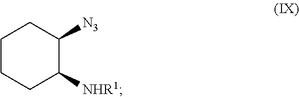

and
(h) contacting a compound of Formula (IX) wherein R¹ is —C(O)OC(CH₃)₃ with a reducing agent to provide the compound of Formula (II).

In one group of embodiments, the base in part (d) is sodium hydroxide. In one group of embodiments, the reducing agent in part (e) is H₂ and Pd(OH)₂. In one group of embodiments, the alkylsulfonylhalide in part (f) is methanesulfonyl chloride and Y is —OS(O)₂CH₃ in Formula (VIII). In one group of embodiments, a crown ether such as 15-crown-5 is added in part (g). In one group of embodiments, the reducing agent in part (h) is H₂ and Pd on C. In one group of embodiments, the compound of Formula (II) has an enantiomeric excess of at least 98% e.e.

In one group of embodiments, wherein the compound of Formula (Ib) is prepared by contacting a compound of Formula (Ie) with a compound of Formula (If) or a salt thereof and a base:

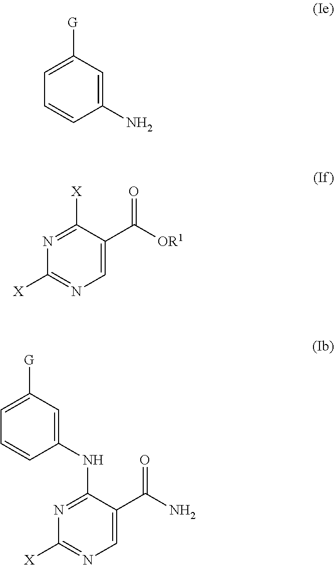

wherein each X is independently, a leaving group.

In one group of embodiments, wherein the compound of Formula (Id) is prepared by contacting a compound of Formula (Ie) with a compound of Formula (If) or a salt thereof and a base:

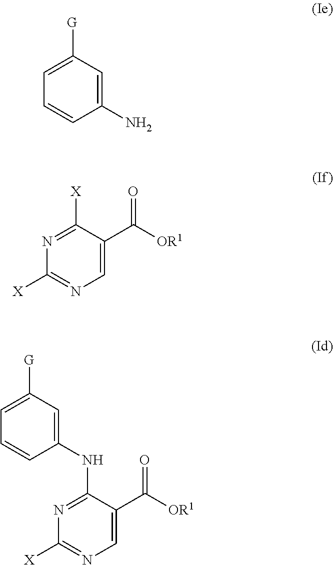

wherein each X is independently, a leaving group and R¹ is C₁-C₈alkyl.

In some embodiments, X is chloro. In some embodiments, R¹ is ethyl. In some embodiments, the organic base is an alkylamine such as triethylamine In some embodiments the reaction is carried out in an alcoholic solvent such as absolute ethanol. An equivalent amount or a slight excess of (Ie) can be used to react with (If) to displace halogen X in the presence of about 5 to about 15 or about 10 equivalents of the organic base. In some embodiments a solution of (If) and base is cooled to 20° C. or less followed by addition of (Ie). The reaction is stirred until complete and the product (Ib) can be washed such as with an ether to remove unreacted (Ie) and other impurities and also to displace water and to make the solid easier to dry. Details of a procedure and an example for the preparation of (Ib) where R² is methyl, R¹ is ethyl, and G is 1H-1,2,3-triazol-2-yl is given in Process Example 1 and Synthesis Example 1.

In one group of embodiments, the compound of Formula (Ia) is

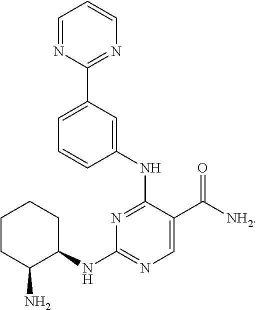 [SMILES: NC(=O)c1cnc(N[C@@H]2CCCC[C@@H]2N)nc1Nc1cccc(-c2ncccn2)c1]

In one group of embodiments, the compound of Formula (Ia) is

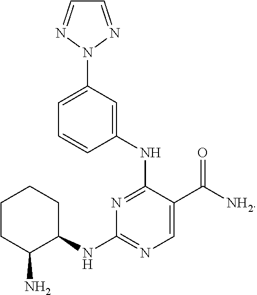 [SMILES: NC(=O)c1cnc(N[C@@H]2CCCC[C@@H]2N)nc1Nc1cccc(-n2nccn2)c1]

In one group of embodiments, the compound of Formula (Ie) is a compound of Formula (X):

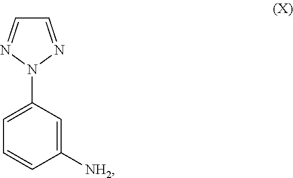 [SMILES: Nc1cccc(-n2nccn2)c1]

wherein said compound is prepared by
(a) contacting a compound of Formula (XI) wherein W is halo with 1H-1,2,3-trizole to provide a compound of Formula (XII)

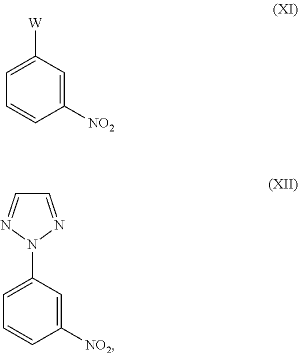

and
(b) contacting a compound of Formula (XII) with a reducing agent to form a compound of Formula (X).

In one group of embodiments, W is fluoro and the reducing agent is H₂ and Pd on carbon.

In one group of embodiments, the invention provides a process for preparing a compound of Formula (II):

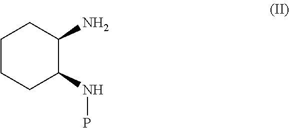

wherein P is a protecting group, the process comprising:
(a) contacting racemic mixture (IV) with D-mandelic acid

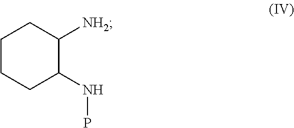

(b) isolating D-mandelic acid salt (V):

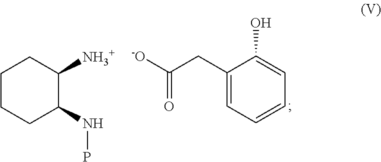

and
(c) contacting the D-mandelic acid salt (V) with base to provide the compound of Formula (II).

In one group of embodiments, the compound of Formula (II) has an enantiomeric excess of at least 98% e.e.

In one group of embodiments, the base is potassium carbonate.

In one group of embodiments, the compound of Formula (IV) is prepared by
(a) contacting 1,2-cyclohexanediamine with DL-tartaric acid to provide a tartaric acid salt of trans-1,2-cyclohexanediamine; and
(b) removing cis-1,2-cyclohexanediamine from the tartaric acid salt of trans-1,2-cyclohexanediamine and isolating cis-1,2-cyclohexanediamine (III):

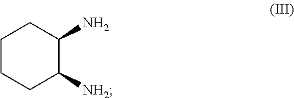

and
(c) contacting cis-1,2-cyclohexanediamine (III) with an acid and di-tert-butyl dicarbonate to provide a racemic mixture of tert-butyl-(1S,2R)-2-aminocyclohexylcarbamate (IV).

In one group of embodiments, the invention provides a process for preparing a compound of Formula (IIa):

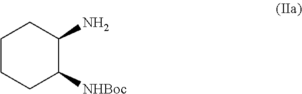 [SMILES: CC(C)(C)OC(=O)N[C@H]1CCCC[C@H]1N]

wherein Boc is —C(O)OC(CH₃)₃, the process comprising:
(a) contacting cyclohexene oxide with benzylamine to provide a racemic mixture of a compound of Formula (VI) wherein R¹ is —CH₂Ph:

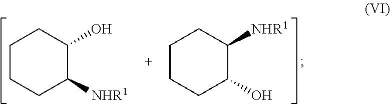

(b) contacting the racemic mixture (VI) with D-mandelic acid;
(c) isolating D-mandelic acid salt (VII) wherein R¹ is —CH₂Ph:

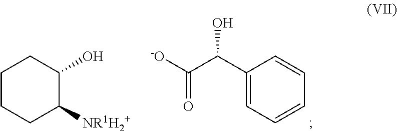

(d) contacting salt (VII) with base to provide a compound of Formula (VIII) wherein Y is OH and R¹ is —CH₂Ph:

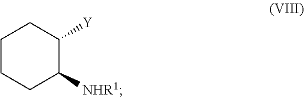

(e) contacting a compound of Formula (VIII) wherein Y is OH and R¹ is —CH₂Ph with a reducing agent and then with di-tert-butyl dicarbonate to provide a compound of Formula (VIII) wherein Y is OH and R¹ is —C(O)OC(CH₃)₃;
(f) contacting a compound of Formula (VIII) wherein Y is OH and R¹ is —C(O)OC(CH₃)₃ with an alkylsulfonylhalide to form a compound of Formula (VIII) wherein Y is —OS(O)₂alkyl and R¹ is —C(O)OC(CH₃)₃;
(g) contacting a compound of Formula (VIII) wherein Y is —OS(O)₂C₁₋₈alkyl and R¹ is —C(O)OC(CH₃)₃ with MN₃ where M is selected from the group consisting of Li, K, or Na to form a compound of Formula (IX) wherein R¹ is —C(O)OC(CH₃)₃:

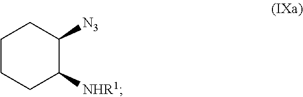

and
(h) contacting a compound of Formula (IXa) wherein R¹ is —C(O)OC(CH₃)₃ with a reducing agent to provide the compound of Formula (II).

In one group of embodiments, in step (d) the base is sodium hydroxide.

In one group of embodiments, in step (e) the reducing agent is H₂ and Pd(OH)₂.

In one group of embodiments, in step (f) the alkylsulfonylhalide is methanesulfonyl chloride and Y is —OS(O)₂CH₃ in Formula (VII).

In one group of embodiments, in step (g) a crown ether such as 15-crown-5 is added.

In one group of embodiments, in step (h) the reducing agent is H₂ and Pd on C.

In one group of embodiments, the compound of Formula (II) has an enantiomeric excess of at least 98% e.e.

In one group of embodiments, a compound of Formula (Id) is reacted with formamide and an alkoxide to form a compound of Formula (Ib)

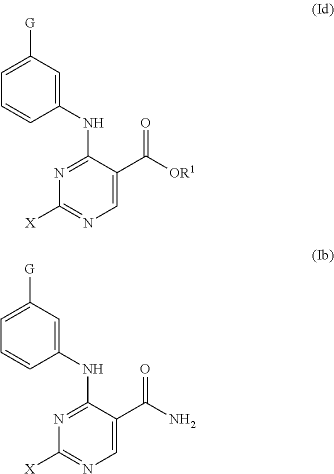

where R¹ is C₁-C₈alkyl. Alkoxides can include sodium and potassium alkoxides. In some embodiments, the alkoxide is sodium ethoxide. The reaction can be carried out in a polar solvent such as dimethylformamide. In one group of embodiments, a solution containing (Id) and excess formamide is cooled to 20° C. or less followed by addition of alkoxide. The reaction is stirred until complete and the reaction is quenched with water, filtered, and washed with water and an ether such as methyl-tert-butyl ether. Details of a procedure and an example for the preparation of (Ib) where X is —SCH₃ and G is 1H-1,2,3-triazol-2-yl is given in Process Example 2 and Synthesis Example 2.

In one group of embodiments, a compound of Formula (Ib) is reacted with tert-butyl-(1S,2R)-2-aminocyclohexylcarbamate (II) to form a compound of Formula (Ic):

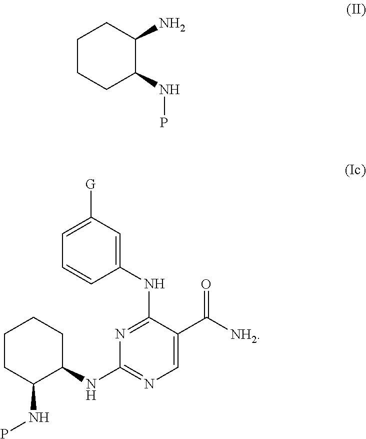

In certain embodiments, group X of compound Ib is a thioether such as —SCH₃, which is contacted with an oxidizing agent before the compound of Formula (Ib) is contacted with a compound of Formula (II). Oxidizing agents include peracids such as meta-chloroperbenzoic acid and the oxidation reaction can be carried out in a polar solvent such as NMP (n-methylpyrrolidone). In one group of embodiments, at least two equivalents of the oxidizing agent is reacted with (Ib) to provide the corresponding sulfoxide and sulfone intermediates which can be filtered and dried. In one group of embodiments, the crude intermediates are reacted with a slight excess of cyclohexyl amine (II) in a polar solvent such as dimethylformamide and in the presence of an organic base such as an alkyl amine Details of a procedure and an example for the preparation of (Ic) where G is 1H-1,2,3-triazol-2-yl is given in Process Example 3 and Synthesis Example 3.

In one group of embodiments, a compound of Formula (Ic) is reacted with acid for carbamate removal and then with base to provide a compound of Formula (Ia) as a free base. In some embodiments, the acid is HCl. In some embodiments, HCl gas is bubbled into an ethyl acetate solution containing (Ic) followed by addition of sodium hydroxide. In some embodiments the free base (Ia) is reacted with acetic acid to provide (Ia) as its acetate salt. Details of a procedure and an example for the preparation of (Ia) and its acetate salt where G is 1H-1,2,3-triazol-2-yl is given in Process Example 4 and Synthesis Example 4.

An example of a process for further purification of the acetate salt of (Ia) is provided in Process Example 5 and Synthesis Example 5. Acetate salt of (Ia) is treated with base such as aqueous sodium hydroxide. The free base of (Ia) is extracted from the reaction mixture and then treated with acetic acid to re-form the acetate salt.

In one group of embodiments, intermediate (II) is prepared by a chiral resolution, wherein 1,2-cyclohexanediamine (III) is contacted with DL-tartaric acid to provide a tartaric acid salt of trans-1,2-cyclohexanediamine The DL-tartaric acid can be added dropwise to 1,2-cyclohexanediamine refluxing in an alcoholic solvent such as ethanol. Following addition, the reaction mixture can be stirred at ambient temperature for 3 to 14 hours or until the reaction is complete. The suspension that forms during the reaction is filtered and leaving cis-1,2-cyclohexanediamine in the filtrate. In one group of embodiments, one equivalent of acid such as HCl is added to the filtrate. The filtrate can be cooled to about 0-10° C. or about 0-5° C. prior to the addition of acid. The acidified filtrate can be stirred from 1 to 4 hrs or for 2 hours followed by addition of di-tert-butyl dicarbonate. The mixture is further stirred for 3 to 14 hours at ambient temperature or until the reaction is complete to provide a racemic mixture of tert-butyl-(1S,2R)-2-aminocyclohexylcarbamate (IV).

In one group of embodiments, the racemic mixture (IV) is contacted with 0.5 equivalents of D-mandelic acid to form the mandelic acid salt (V):

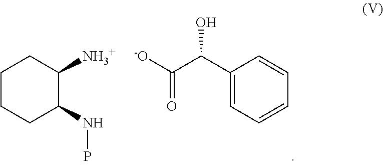

In one group of embodiments, the solution can be stirred for 3 to 14 hours at ambient temperature to form (V) as a solid that is then filtered. The solid (V) can be further purified by recrystallization such as from isopropyl alcohol.

In one group of embodiments, treatment of salt (V) with at least one equivalent of base in an organic solvent provides tert-butyl-(1S,2R)-2-aminocyclohexylcarbamate (II). Suitable bases include inorganic bases such as K₂CO₃. Suitable organic solvents include ethyl acetate, and the reaction can be formed under ambient temperatures or from 20-30° C. In some embodiments, the process provides salt (V) or intermediate (If) having at least 98% or 99% e.e.

The opposite (1R,2S) enantiomer salt of (V) can be obtained from the mother liquor resulting from the filtration and treated with base to give the enantiomer of (II).

In one group of embodiments, intermediate (IIa) is prepared according to Scheme I.

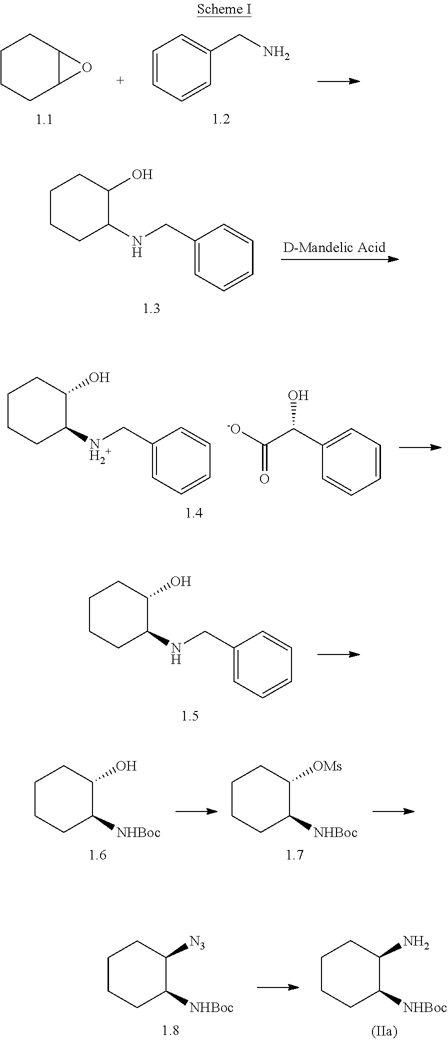

Cyclohexene oxide 1.1 is reacted with benzylamine 1.2 to give amine 1.3. The reaction can be carried out in water and refluxed after addition of the benzylamine Following workup and isolation, racemic trans amine 1.3 can be purified by recrystallization such as from heptane.

The purified amine is contacted with 0.5 equivalents of D-mandelic acid (also known as (R)-mandelic acid) in an organic solvent such as ethyl acetate to give the desired isomer 1.4 as a white precipitate that is filtered and washed with an organic solvent.

Treatment of 1.4 with base such as NaOH in an organic solvent such as tert-butyl methyl ether gives the free base 1.5.

Deprotection of benzyl amine proceeds by reaction with any number of known reducing agents such as hydrogen using catalytic amounts of an appropriate transition metal. In one group of embodiments, the reducing agent is H₂/Pd(OH)₂ and the reaction is carried out in methanol. Upon completion the reaction is then filtered and the free amine reacted with di-t-butyl dicarbonate in the presence of an organic base such as triethylamine to form the BOC protected amine 1.6.

Displacement of the alcohol proceeds by converting the alcohol to a leaving group. In one group of embodiments the alcohol is treated with an alkylsulfonyl halide such as methanesulfonyl chloride to form mesylate 1.7. In one group of embodiments, the reaction is carried out in an organic solvent such as dichloromethane at a temperature of between 0-5° C. Mesylate 1.7 is reacted with NaN₃ to give azide 1.8. In one group of embodiments, the reaction is carried out in a polar solvent such as dimethylformamide at a temperature of between 80-140° C. A crown ether can also be added to the reaction make the azide anion more nucleophilic. Suitable crown ethers for use in the reaction include 15-crown-5. Azide 1.8 can be purified by silica gel chromatography.

Treatment of azide 1.8 with a reducing agent such as hydrogen using catalytic amounts of an appropriate transition metal gives amine (IIa). In one group of embodiments, the reducing agent is H₂/Pd/C. Further details of the preparation of (IIa) from 1.1 and 1.2 is given in Synthesis Example 7.

In one group of embodiments, triazole (Xa) is prepared according to Scheme II.

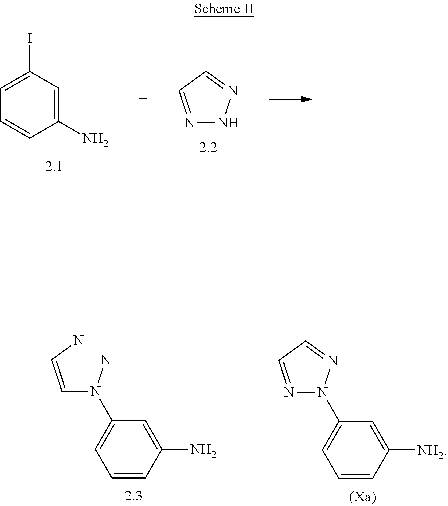 [SMILES: Nc1cccc(I)c1 ; c1cn[nH]n1 ; N/C=C\N(N)c1cccc(N)c1 ; Nc1cccc(-n2nccn2)c1]

3-Iodoaniline 2.1 is reacted with triazole 2.2 in the presence of CuI, a phosphate salt, and an amine such as ethylenediamine The reaction can be conducted in a polar solvent such as a mixture of dioxane and dimethyl sulfoxide (DMSO) at elevated temperature such as under refluxing conditions. The resulting mixture of regioisomers with amine (Xa) as the major product can be seperated by silica gel chromatography. An example of the preparation of (Xa) using this process is giving in Synthesis Example 8.

In one group of embodiments, triazole (Xa) is prepared according to Scheme III.

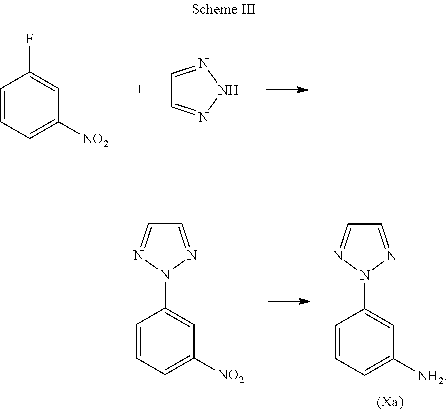 [SMILES: O=[N+]([O-])c1cccc(F)c1 ; c1cn[nH]n1 ; O=[N+]([O-])c1cccc(-n2nccn2)c1 ; Nc1cccc(-n2nccn2)c1]

1-Fluoro-3-nitrobenzene is reacted with 1H-1,2,3-triazole in a polar solvent in the presence of base. In some embodiments the solvent is N-methyl-2-pyrrolidone (NMP). In some embodiments, the base is Cs₂CO₃. In some embodiments the resulting mixture of regioisomers is separated by silica gel chromatography. The 2-(3-nitrophenyl)-2H-1,2,3-triazole is exposed to reducing conditions such as by reaction with H₂ and Pd/C to form amine (Xa). An example of the preparation of (Xa) following such a procedure is given in Synthesis Examples 9 and 10.

In another embodiment, 1-fluoro-3-nitrobenzene is reacted with 1H-1,2,3-triazole in DMF and with NaH as the base to give a 1.4:1 mixture of 2-(3-nitrophenyl)-2H-1,2,3-triazole and 1-(3-nitrophenyl)-1H-1,2,3-triazole. The mixture is then exposed to reducing conditions such as by reaction with H₂ and Pd/C. The reaction is then filtered and the product is recrystallized from methanol to give the desired 3-(2H-1,2,3-triazol-2-yl)aniline.

In another group of embodiments, product (VIa) is prepared according to Scheme IV.

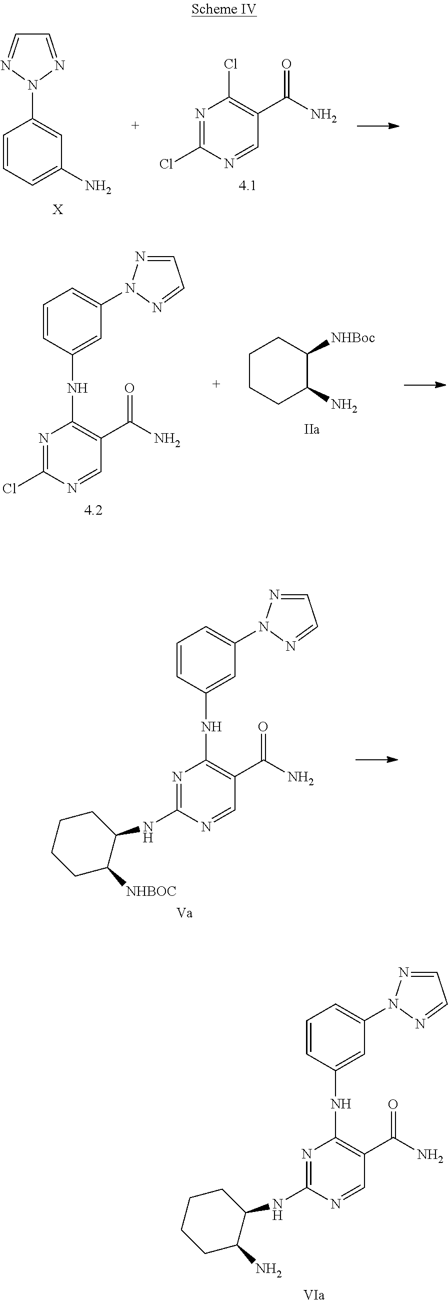

The route is three steps and a salt formation step. The first step is a regioselective displacement of the 4-chloro of 4.1. The isolated yield of 4.2 for this step is greater than 95%.

In the second step the amine of IIa displaces the 2-chloro of 4.2 to afford Va which is not isolated but treated with HCl to de-protect and VIa is isolated as the di-hydrochloride salt in 85% yield. In one embodiment, the di-hydrochloride salt of VIa is isolated after Boc-deprotection and converted to the desired salt form. In one embodiment, the salt is an acetate salt.

In an alternate embodiment, the amine of compound X displaces the 4-chloro of 5.1 to afford IIIa as shown in Scheme V below. IIIa is converted to the amide IVa and the thiomethyl group is oxidized. The amine of IIa then displaces the leaving group at the 2-position of 5.2 and/or 5.3 to provide Va.

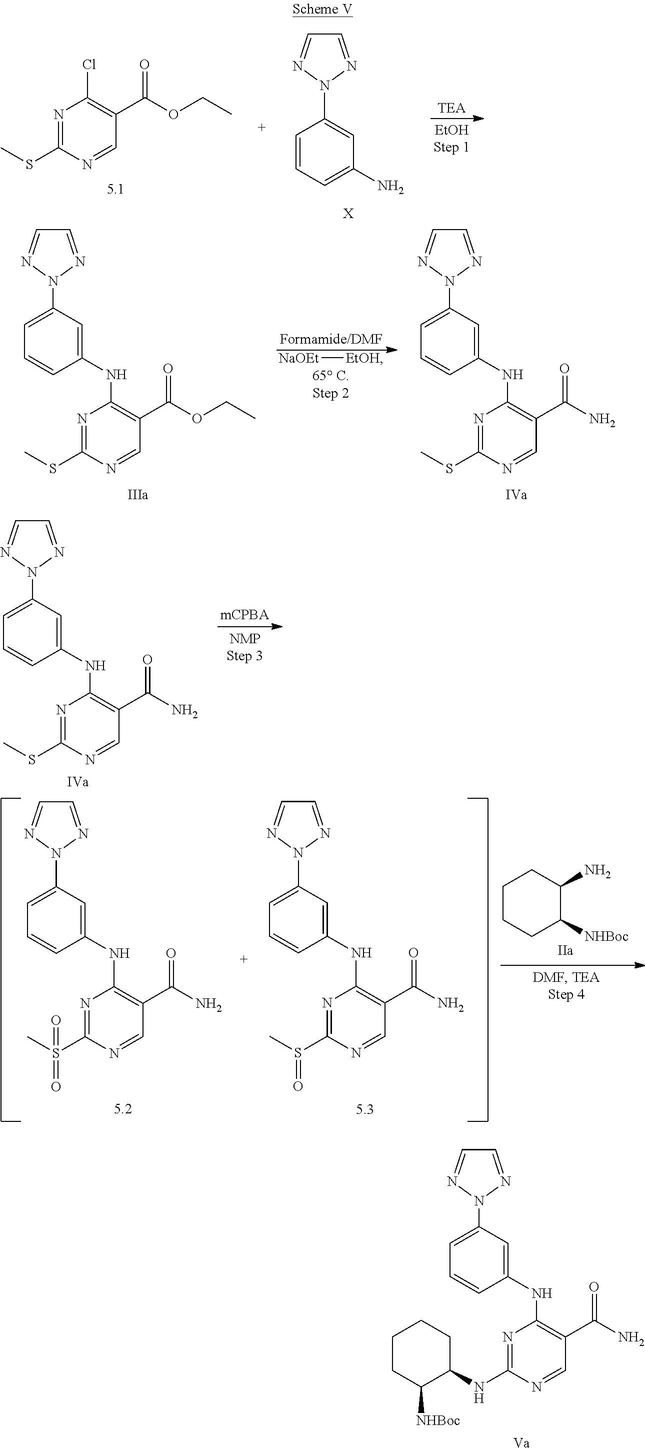

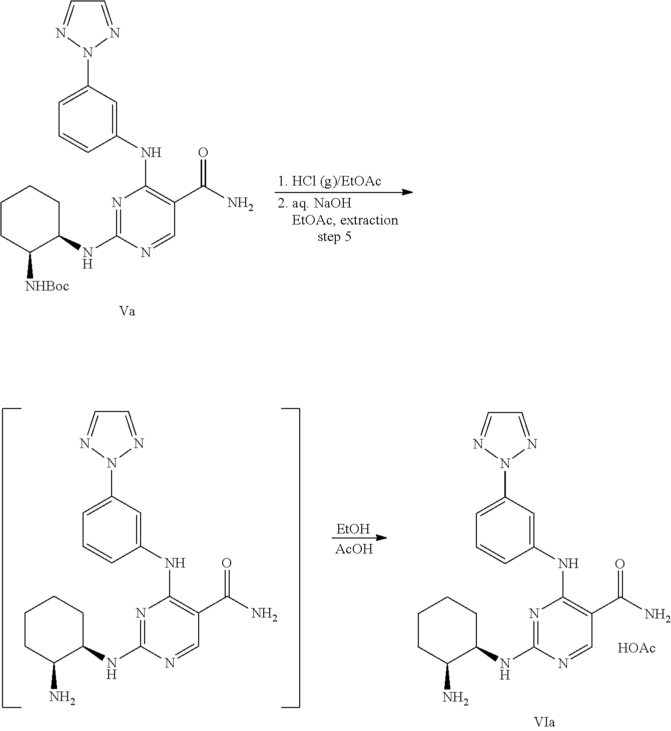

Accordingly, contemplated within the scope of embodiments presented herein is the use of any suitable leaving group at the 2-position (LG¹) and the 4-position (LG²) of the pyrimidine ring as shown in Scheme VI below. Examples of leaving groups include, and are not limited to, halo (e.g., bromo, chloro, iodo), alkoxy, thioalkoxy, alkylsulfinyl, alkylsulfonyl, haloalkylsulfonyl (e.g., triflate) or any other suitable leaving groups. In some embodiments, LG¹ and LG² are the same. In other embodiments, LG¹ and LG² are not the same. It is understood that the compounds may be used as either free bases or salts for any reaction described herein.

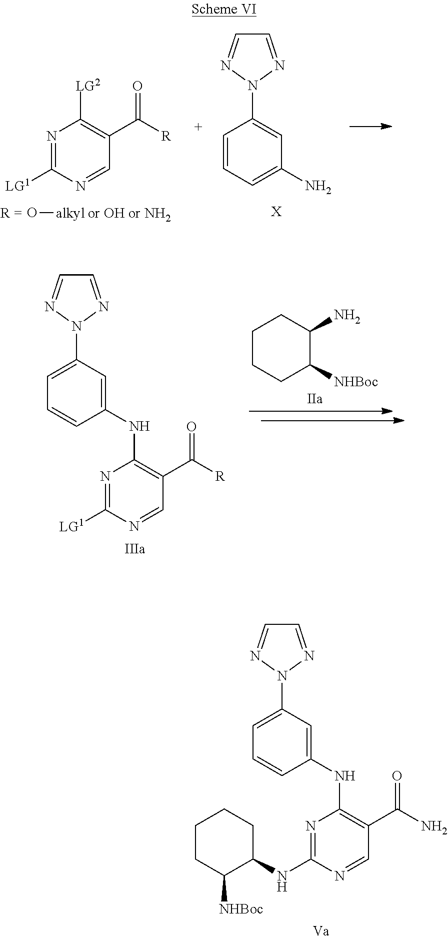

It is understood that in another group of embodiments, any of the above embodiments may also be combined with other embodiments listed herein, to form other embodiments of the invention. Similarly, it is understood that in other embodiments, listing of groups includes embodiments wherein one or more of the elements of those groups is not included.

### EXAMPLES

The following examples are offered to illustrate, but not to limit, the claimed invention. Variations such as in the temperature, reaction time, amounts, etc., can be adjusted as appropriate depending on the scale of the reaction, and such modifications are within the skill of one of skill in the art.

### General Methods

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1967-2004, Volumes 1-22; Rodd's Chemistry of Carbon Compounds, Elsevier Science Publishers, 1989, Volumes 1-5 and Supplementals; and Organic Reactions, Wiley & Sons: New York, 2005, Volumes 1-65. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C. to about 75° C.

Referring to the examples that follow, compounds of the present invention were synthesized using the methods described herein, or other methods, which are well known in the art.

The compounds and/or intermediates were characterized by high performance liquid chromatography (HPLC) using a Waters Alliance chromatography system with a 2695 Separation Module (Milford, Mass.). The analytical columns were C-18 SpeedROD RP-18E Columns from Merck KGaA (Darmstadt, Germany). Alternately, characterization was performed using a Waters Unity (UPLC) system with Waters Acquity UPLC BEH C-18 2.1 mm×15 mm columns. A gradient elution was used, typically starting with 5% acetonitrile/95% water and progressing to 95% acetonitrile over a period of 5 minutes for the Alliance system and 1 minute for the Acquity system. All solvents contained 0.1% trifluoroacetic acid (TFA). Compounds were detected by ultraviolet light (UV) absorption at either 220 or 254 nm. HPLC solvents were from EMD Chemicals, Inc. (Gibbstown, N.J.). In some instances, purity was assessed by thin layer chromatography (TLC) using glass backed silica gel plates, such as, for example, EMD Silica Gel 60 2.5 cm×7.5 cm plates. TLC results were readily detected visually under ultraviolet light, or by employing well known iodine vapor and other various staining techniques.

Mass spectrometric analysis was performed on one of two Agilent 1100 series LCMS instruments with acetonitrile/water as the mobile phase. One system using TFA as the modifier and measures in positive ion mode [reported as MH+, (M+1) or (M+H)+] and the other uses either formic acid or ammonium acetate and measures in both positive [reported as MH+, (M+1) or (M+H)+] and negative [reported as M−, (M−1) or (M−H)−] ion modes.

Nuclear magnetic resonance (NMR) analysis was performed on some of the compounds with a Varian 400 MHz NMR (Palo Alto, Calif.). The spectral reference was either TMS or the known chemical shift of the solvent.

The purity of some of the invention compounds is assessed by elemental analysis (Robertson Microlit, Madison N.J.).

Melting points are determined on a Laboratory Devices Mel-Temp apparatus (Holliston, Mass.).

Preparative separations were carried out using either an Sq16x or an Sg100c chromatography system and prepackaged silica gel columns all purchased from Teledyne Isco, (Lincoln, Nebr.). Alternately, compounds and intermediates were purified by flash column chromatography using silica gel (230-400 mesh) packing material, or by HPLC using a C-18 reversed phase column. Typical solvents employed for the Isco systems and flash column chromatography were dichloromethane, methanol, ethyl acetate, hexane, acetone, aqueous hydroxyamine and triethyl amine Typical solvents employed for the reverse phase HPLC were varying concentrations of acetonitrile and water with 0.1% trifluoroacetic acid.

The following process examples detail steps for preparing the indicated intermediates and compounds based on a 1 kg of the indicated starting material. Specific equipment used in the examples include those shown in the table below.

| Equipment | | | | |
|---|---|---|---|---|
| type | Material | Specification | Agitator type | Manufacturer |
| Reactor A/C | Glass-lined | 800 liters | 3 blades | R&M Italia |
| (Jacketed | carbon steel | | Retreat curve | |
| reactor) | | | | |
| Reactor B | Hastelloy C22 | 400 liters | Bottom: Flat- | Walker |
| (Jacketed | | | blade turbine | Stainless Steel |
| reactor) | | | Top: Pitched- | Equipment |
| | | | blade Turbine | |
| Manually- | Hastelloy C22 | 0.3 m²/105 | Manual | Thompson |
| agitated | | liters | | Filters |
| oyster-style | | | | |
| filter | | | | |
| Vacuum tray | Chamber: SS | 1.86 m² | — | Bucher-Guyer |
| dryer | 304 Halar-lined | | | |
| | Shelves: | | | |
| | Hastelloy C22 | | | |
| | Trays: | | | |
| | Hastelloy C22 | | | |

### Process Example 1

### Preparation of ethyl 4-(3-(2H-1,2,3-triazol-2-yl)phenylamino)-2-(methylthio)pyrimidine-5-carboxylate

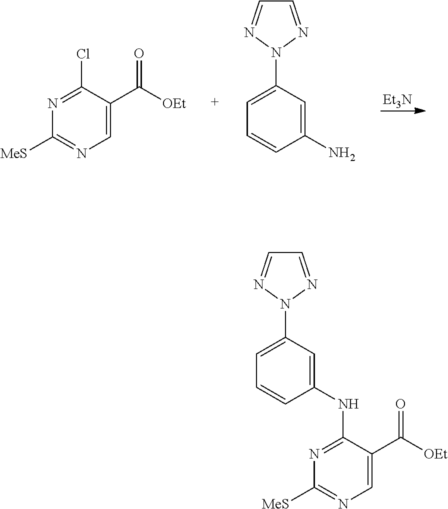 [SMILES: "Et3N" ; CCOC(=O)c1cnc(SC)nc1Cl ; Nc1cccc(-n2nccn2)c1 ; CCOC(=O)c1cnc(SC)nc1Nc1cccc(-n2nccn2)c1]

Reactor A was charged with solid ethyl 4-chloro-2-methylthio-5-pyrimidine carboxylate (1.00 kg). Reactor A was then charged with absolute ethanol (5.00 L, 4.0 kg). Reactor A was then charged with triethylamine (0.62 L, 0.45 kg). The content of Reactor A was then cooled to about 20° C. Reactor A was then charged 3-amino-N-phenyl-triazole (0.70 kg). The reaction mixture was slowly heated to 30-40° C. over about 1 hour to give an off white suspension. The contents of Reactor A was stirred between 30-40° C. for about 6 hours. 8) Sample solution for ion pair chromatography for less than 1% (area) by HPLC of ethyl 4-chloro-2-methylthio-5-pyrimidine carboxylate. If the sample result complies with criterion proceed to the next step, otherwise agitate for an additional 2 hours between 30-40° C. and sample again. The reaction mixture generally turns into almost unmixable slurry toward the end of the reaction. The contents of Reactor A was cooled to about 20° C. Tap water (8.00 L, 8.00 kg) was charged into Reactor B and the temperature of the contents of Reactor B was adjusted to about 15° C. The contents of Reactor B was charged into Reactor A, while maintaining the temperature at about 15° C. in Reactor A. The contents of Reactor A was stirred for about 30 minutes at about 15° C. The contents of Reactor A was filtered using a filter cloth of about 8 μm or smaller to accommodate the particle size, or an oyster-style filter with a 3-5 μm polypropylene filter cloth. Reactor B was charged with tap water (10.00 L, 10.00 kg) and half the contents of Reactor B was charged to the filter to wash the solids. The remaining contents of Reactor B was charged to the filter to wash the solids. Reactor B was charged with methyl tert-butyl ether (3.00 L, 2.22 kg). The contents of Reactor B was then charged to the filter to wash the solids. The contents of the filter was dried under vacuum such as in a vacuum tray dryer at about 55° C. for about 12 hours, until the water (by Karl Fischer) is <1% w/w.

### Process Example 2

### Preparation of 4-(3-(2H-1,2,3-triazol-2-yl)phenylamino)-2-(methylthio)pyrimidine-5-carboxamide

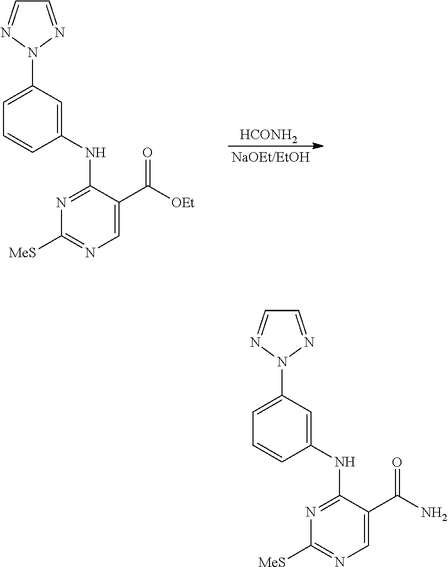 [SMILES: CCOC(=O)c1cnc(SC)nc1Nc1cccc(-n2nccn2)c1 ; "HCONH2" ; CSc1ncc(C(N)=O)c(Nc2cccc(-n3nccn3)c2)n1]

Reactor A was charged with ethyl 4-(3-(2H-1,2,3-triazol-2-yl)phenylamino)-2-(methylthio)pyrimidine-5-carboxylate (1.00 kg). Reactor A was then charged with DMF (4.00 L, 3.78 kg) and then formamide (1.00 L, 1.13 kg). The temperature in Reactor A was adjusted to about 20° C. Reactor A was then charged with sodium ethoxide 21% solution (1.60 L, 1.39 kg) and the contents was stirred at 60-70° C. for about 2 hours. Reactor A was sampled by HPLC until less than 1% area starting material remained. If more than 1% starting material remained, the reaction mixture was charged with sodium ethoxide 21% solution (0.100 L, 0.087 kg) and agitated for about 2 hours at 60-70° C. and sampled again, as necessary. The contents of Reactor A was cooled to about 20° C. Then Reactor A was charged with tap water (15.00 L, 15.00 kg) while keeping the temperature at about 30° C. The contents of Reactor A was cooled to about 10° C. and agitated for about 4 hours at about 10° C. The contents of Reactor A was filtered through Filter A. Reactor A was charged with tap water (5.00 L) and the contents of Reactor A was transferred to Filter A. Reactor A was then charged with tap water (5.00 L) and the contents of Reactor A was transferred to Filter A. Reactor A was charged with tap water (5.00 L, 5.00 kg) and the contents of Reactor A was transferred to Filter A. Reactor A was then charged with MTBE (3.00 L, 2.22 kg) and the contents of Reactor A transferred to Filter A. Reactor A was charged with MTBE (3.00 L, 2.22 kg) and the contents of Reactor A was transferred to Filter A. The contents of Filter A was dried under vacuum such as in a vacuum tray dryer at about 55° C. for about 12 hours, until water by Karl Fischer is <1.0% w/w.

### Process Example 3

### Preparation of tert-butyl(1S,2R)-2-(4-(3-(2H-1,2,3-triazol-2-yl)phenylamino)-5-carbamoylpyrimidin-2-ylamino)cyclohexylcarbamate

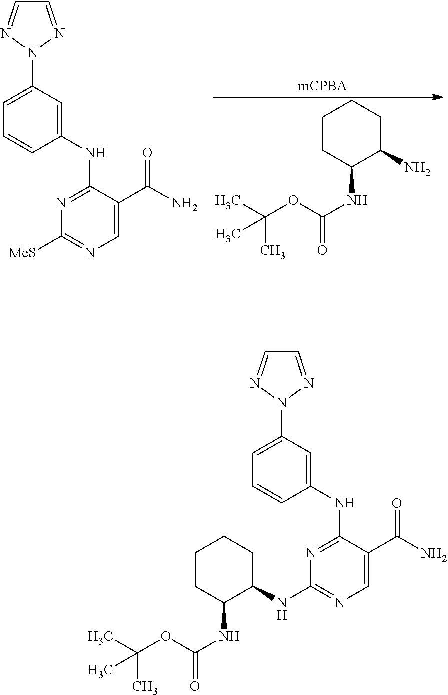 [SMILES: CSc1ncc(C(N)=O)c(Nc2cccc(-n3nccn3)c2)n1 ; CC(C)(C)OC(=O)N[C@H]1CCCC[C@H]1N ; CC(C)(C)OC(=O)N[C@H]1CCCC[C@H]1Nc1ncc(C(N)=O)c(Nc2cccc(-n3nccn3)c2)n1]

Reactor A was charged with 1.00 kg 4-(3-(2H-1,2,3-triazol-2-yl)phenylamino)-2-(methylthio)pyrimidine-5-carboxamide. Reactor A was charged with NMP (n-methylpyrrolidone, 6.00 L, 6.18 kg) and the contents of Reactor A was cooled to about 5° C. Reactor A was charged with 3-chloroperbenzoic acid (1.13 kg) in portions while keeping the temperature at about 35° C. (addition can be exothermic). The contents was stirred at 30-40° C. for about 2 hours. Reactor A was sampled by HPLC until the area of starting material was <1%. If the starting material was 1% or more, 3-chloroperbenzoic acid (0.10 kg) was added and the reaction mixture was agitated for an additional 2 hours at 30-40° C. and sampled. The contents of Reactor A was cooled to about 5° C. Reactor A was charged with DCM (dichloromethane, 10.00 L, 13.30 kg) and agitate for about 1 hour at about 5° C. The contents of Reactor A was Tilted through Filter A such as through an oyster type filter using a 3-5 μm polypropylene filter cloth. Reactor A was charged with DCM (dichloromethane, 3.00 L, 3.99 kg). The contents of Reactor A was filtered through Filter A. Reactor A was charged with DCM (3.00 L, 3.99 kg). The contents of Reactor A was filtered through Filter A. The contents of Filter A was dried under vacuum at about 55° C. for about 24 hours. Reactor A was charged with the dried intermediate. Reactor A was then charged with (1S,2R)-1-Boc-1,2-diaminocyclohexane (0.69 kg, broken into small lumps). Reactor A was charged with triethylamine (0.51 L, 0.37 kg) and then DMF (4.00 L, 3.78 kg). The contents of Reactor A was agitated at 60-70° C. for about 2 hours.

Reactor A was sampled for <1% area sulfone and sulfoxide by HPLC. If the starting material was 1% or more, the reaction mixture was charged with (1S,2R)-1-Boc-1,2-diaminocyclohexane (0.05 kg) and agitated at 60-70° C. for an additional 1 hour and sample again. The contents of Reactor A was cooled to about 30° C. Reactor B was charged with tap water (15.00 L, 15.00 kg) and the contents of Reactor B was cooled to about 20° C. The contents of Reactor A was slowly charged to Reactor B, keeping the temperature at about 30° C. and vigourously agitated to help precipitate out the solids uniformly. Reactor A was charged with DMF (0.5 L, 0.47 kg). The contents of Reactor A was charged to Reactor B. Reactor A was charged with DMF (0.5 L, 0.47 kg) and the contents of Reactor A was transferred to Reactor B. The contents of Reactor B was agitated for about 1 hour at about 30° C. The contents of Reactor B was filtered through Filter A (e.g. oyster-style filter using 3-20 μm polypropylene filter cloth). Filtration can be performed in multiple parts for a thick slurry. Pressure was gradually applied (e.g. up to 0.5 barg) and when mother liquor stopped coming out, mild vacuum was applied (approximately 0.40 barg) in combination with the pressure (approximately 0.5 barg). Reactor B was charged with tap water (3.00 L, 3.00 kg) and the contents of Reactor B was filtered through Filter A. Reactor B was charged with tap water (3.00 L, 3.00 kg). The contents of Reactor B was filtered through Filter A. Filter A was charged with n-Heptane (3.00 L, 2.04 kg) and n-Heptane (3.00 L, 2.04 kg). The contents of the filter was dried under vacuum about 55° C. for about 12 hours, until water by Karl Fischer is <1% w/w. Drying can be carried out under vacuum such as in a vacuum tray dryer.

### Process Example 4

### Preparation of 4-(3-(2H-1,2,3-triazol-2-yl)phenylamino)-2-((1R,2S)-2-aminocyclohexylamino)pyrimidine-5-carboxamide and its acetic acid salt

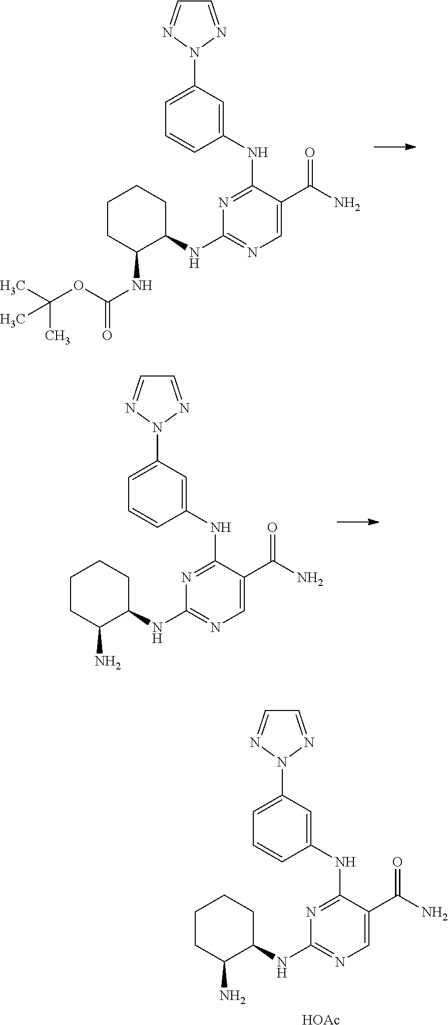 [SMILES: CC(C)(C)OC(=O)N[C@H]1CCCC[C@H]1Nc1ncc(C(N)=O)c(Nc2cccc(-n3nccn3)c2)n1 ; NC(=O)c1cnc(N[C@@H]2CCCC[C@@H]2N)nc1Nc1cccc(-n2nccn2)c1 ; NC(=O)c1cnc(N[C@@H]2CCCC[C@@H]2N)nc1Nc1cccc(-n2nccn2)c1 ; "HOAc"]

Reactor A was charged with 1.00 kg tert-butyl (1S,2R)-2-(4-(3-(2H-1,2,3-triazol-2-yl)phenylamino)-5-carbamoylpyrimidin-2-ylamino)cyclohexylcarbamate. Reactor A was then charged with ethyl acetate (15.00 L, 13.43 kg). The contents of Reactor A was stirred at 20-30° C. for about 0.5 hours. During this step the solids mostly dissolved followed by the precipitation of a thick slurry. The contents of Reactor A was cooled to 0-10° C. Reactor A was charged with HCl gas (0.50 kg) at about 15° C. (the addition is exothermic). The contents of Reactor A was stirred at 20-30° C. for about 2 hours. The contents of Reactor A can become very thick and difficult to agitate; so additional charges of EtOAc may be used. Reactor A was sampled until there was <1% area starting material relative to product by HPLC. If 1% or more starting material remained, the reaction mixture was charged with HCl gas (0.10 kg) and agitated for about 2 hours at 20-30° C. and sampled again. The contents of Reactor A was distilled at about 45° C. under vacuum until a volume of about 5 L remained. Reactor A was then charged with ethyl acetate (5.00 L, 4.48 kg). The contents of reactor A was distilled at about 45° C. under vacuum until a volume of about 5 L remains. Reactor A was then charged with absolute ethanol (2.50 L, 1.97 kg). The contents in Reactor A was cooled to 0-10° C. and Reactor A was charged with 5N sodium hydroxide solution in deionized water (1.35 L, 1.62 kg) at about 15° C. to adjust pH≧9. The pH was kept at ≧9 for at least 1 hour. Reactor A was charged with deionized water (2.50 L, 2.50 kg;) and the contents of Reactor A was stirred at 25-35° C. for about 0.5 hours. Stirring was stopped and held for about 15 minutes. The phases were separated, and the aqueous phase was collected in Reactor B. The product will be present in the organic (top) phase. The organic phase appears yellow and the aqueous phase is a lighter yellow. Reactor B was charged with ethyl acetate (4.00 L, 3.58 kg). The contents of Reactor B was stirred at 25-35° C. for about 15 minutes. Stirring was stopped and held for about 15 minutes. The phases were separated and the aqueous phase was collected in Reactor C. The aqueous (bottom) phase was a faint yellow, with the organic phase appearing slightly darker. The organic phases were combined in Reactor A and the aqueous phase in Reactor C was transferred to Reactor B. The previous 6 steps were repeated two more times. Reactor A was charged with deionized water packaged in bulk (2.00 L, 2.00 kg). The contents of Reactor A was stirred at 25-35° C. for about 15 minutes.
27) Stop stirring and hold for about 15 minutes.
28) Phase separation; transfer the aqueous phase in Reactor A to waste. The aqueous phase should be on the bottom and appear clear and colorless.
29) Repeat the previous 4 steps 2 times
30) Distill the contents of Reactor A at about 45° C. and under vacuum to remove the solvent until a volume of about 5 L remains. The preferred temperature is about 50° C., if the solution cools to less than 45° C. the product may precipitate out of solution. Reactor A was charged with absolute ethanol (10.00 L, 7.89 kg) and the contents of Reactor A was distilled at about 45° C. to remove the solvent until a volume of about 5 L remains. If the solution cooled to less than 45° C. the product may precipitate out of solution. The previous two steps were repeated (e.g. absolute ethanol warm up, transfer, and start up of distillation). Reactor A was charged with absolute ethanol (3.00 L, 2.37 kg) and the contents were heated to 45-55° C. The contents of Reactor A were polish filtered hrough Filter A to Reactor B. Reactor A was charged with absolute ethanol (2.00 L, 1.58 kg). The contents of Reactor A was filtered through Filter A to Reactor B. Reactor B was charged with acetic acid (0.17 L, 0.18 kg) and the contents of Reactor B was heated to 45-50° C. The product may start to precipitate during the heating. The contents was stirred at 45-50° C. for about 0.5 hours. The product may begin to precipitate out during the stir. The contents of Reactor B was concentrated to about 5 L at about 45° C. under vacuum.
42) To Reactor B, charge ethyl acetate (5.00 L, 4.48 kg). The contents of Reactor B was cooled to about 20° C. The contents of Reactor B was agitated for about 1 hour at about 20° C. The contents of Reactor B was transferred to Filter B (e.g. oyster-style filter using 8 μm polypropylene filter cloth). Reactor B was charged with ethyl acetate (3.00 L, 2.69 kg). The contents of Reactor B was transferred to Filter B to rinse the cake. Reactor B was charged with ethyl acetate (3.00 L, 2.69 kg). The contents of Reactor B was transferred to Filter B. Reactor B was charged with ethyl acetate (3.00 L, 2.69 kg). The contents of Reactor B was transferred to Filter B. The contents of Filter B were dried at about 50° C. for about 12 hrs and sampled for ethanol by GC<28,500 ppm).

### Process Example 5

### Preparation of 4-(3-(2H-1,2,3-triazol-2-yl)phenylamino)-2-((1R,2S)-2-aminocyclohexylamino)pyrimidine-5-carboxamide acetic acid salt

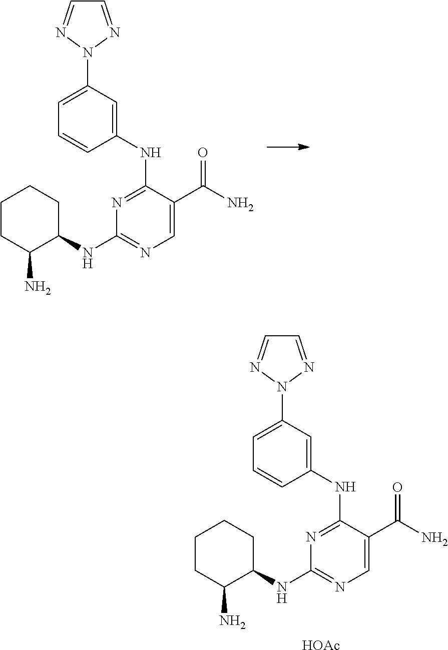 [SMILES: NC(=O)c1cnc(N[C@@H]2CCCC[C@@H]2N)nc1Nc1cccc(-n2nccn2)c1 ; NC(=O)c1cnc(N[C@@H]2CCCC[C@@H]2N)nc1Nc1cccc(-n2nccn2)c1 ; "HOAc"]

Reactor A was charged with 4-(3-(2H-1,2,3-triazol-2-yl)phenylamino)-2((1R,2S)-2-aminocyclohexylamino)pyrimidine-5-carboxamide acetic acid salt (1.00 kg). Reactor A was charged with ethyl acetate (10.00 L, 8.95 kg). Reactor A was charged with absolute ethanol (2.50 L, 1.97 kg). The contents of Reactor A was warmed to 40-50° C. Reactor A was charged with a 5N solution of sodium hydroxide (50% w/w) in deionized water (1.35 L, 1.60 kg) while maintaining a temperature of 40-50° C. and adjusting the pH to ≧9. The pH was kept at ≧9 for at least about 1 hour pH. The reaction mixture went from a slurry to a homogeneous solution. Reactor A was charged with deionized water (2.50 L, 2.50 kg). The temperature of the contents of Reactor A was adjusted to 25-35° C. The contents of Reactor A was stirred at 25-35° C. for about 0.5 hours. The stirring was stopped and held for about 15 minutes. The phases were separated and the aqueous phase was collected in Reactor B. The aqueous phase was a clear and the organic (bottom) phase appeared slightly yellow. Reactor B was charged with ethyl acetate (4.00 L, 3.58 kg). The contents of Reactor B was stirred at 25-35° C. for about 15 minutes. Stirring was stopped and held for about 15 minutes. The phases were separated and the aqueous phase was collected in Reactor C. The aqueous (bottom) phase was clear and the organic was slightly yellow. The organic phases were combined in Reactor A. The aqueous phase in Reactor C was transferred to Reactor B. After the final repetition of this extraction sequence the aqueous phase may be discarded to waste and not to Reactor B. The previous 6 steps were repeated two more times. Reactor A, charge deionized water packaged in bulk (2.00 L, 2.00 kg). The contents of Reactor A was stirred at 25-35° C. for about 15 minutes. Stirring was stopped and held for about 15 minutes. The phases were separated and the aqueous phase was transferred from Reactor A to waste. The aqueous phase was on the bottom and appeared clear and colorless. The previous four steps were repeated 2 times. The contents of Reactor A was distilled at about 45° C. and under vacuum to remove the solvent until a volume of about 5 L remains. The preferred temperature is about 50° C., if the solution cools to less than 45° C. the product may precipitate out of solution. Reactor A was charged with absolute ethanol (10.00 L, 7.89 kg). The contents of Reactor A was distilled under vacuum at about 45° C. to remove the solvent until a volume of about 5 L remains. The preferred temperature is about 50° C., if the solution cools to less than 45° C. the product may precipitate out of solution. The previous two steps were repeated once more. Reactor A was charged with absolute ethanol (2.67 L, 2.11 kg) and the contents of Reactor A was heated to 45-55° C. The contents of Reactor A were polish filtered through Filter A to Reactor B. Ensure that the product is in solution before polish filtration. Reactor A was charged with absolute ethanol (1.00 L, 0.79 kg). Reactor A was charged with deionized water (0.87 L, 0.87 kg). The contents of Reactor A was charged through Filter A to Reactor B. Reactor B was charged with acetic acid (0.19 L, 0.20 kg). The contents of Reactor B was heated to 45-50° C. The product may precipitate out during the heating. The contents was stirred at 45-50° C. for about 0.5 hours. The contents of Reactor B was concentrated under vacuum to about 5 L at about 45° C. Reactor B was charged with ethyl acetate (5.00 L, 4.48 kg). The contents of Reactor B was cooled to about 20° C. The contents of Reactor B was agitated for about 1 hour at about 20° C. The contents of Reactor B was transferred to Filter B. Reactor B was charged with ethyl acetate (3.00 L, 2.69 kg). The contents of Reactor B was transferred to Filter B, to rinse the cake. Reactor B was charged with ethyl acetate (3.00 L, 2.69 kg). The contents of Reactor B ws transferred to Filter B, to rinse the cake. Reactor B was charged with ethyl acetate (3.00 L, 2.69 kg). The contents of Reactor B was charged to Filter B, to rinse the cake. The contents of Filter B was dried at about 40° C. for about 12 hrs and sampled for ethanol by gas chromatography <20,000 ppm.

### Synthesis Example 1

### Preparation of ethyl 4-(3-(2H-1,2,3-triazol-2-yl)phenylamino)-2-(methylthio)pyrimidine-5-carboxylate

The title compound was prepared according to Process Example 1 starting with 25.0 kg of ethyl 4-chloro-2-methylthio-5-pyrimidine, 11.25 kg triethylamine, 100 kg absolute ethanol and 17.5 kg 3-amino-N-phenyl-triazole. A 90.4% yield of the title compound was obtained.

### Synthesis Example 2

### Preparation of 4-(3-(2H-1,2,3-triazol-2-yl)phenylamino)-2-(methylthio)pyrimidine-5-carboxamide

The title compound was prepared in 96.6% according to Process Example 2 starting with 34.6 kg of ethyl 4-(3-(2H-1,2,3-triazol-2-yl)phenylamino)-2-(methylthio)pyrimidine-5-carboxylate, 39.1 kg of formamide, 130.8 kg DMF, and 48.1 kg of a 21% sodium ethoxide solution.

### Synthesis Example 3

### Preparation of tert-butyl(1S,2R)-2-(4-(3-(2H-1,2,3-triazol-2-yl)phenylamino)-5-carbamoylpyrimidin-2-ylamino)cyclohexylcarbamate

The title compound was prepared according to Process Example 3 starting with 30.7 kg of 4-(3-(2H-1,2,3-triazol-2-yl)phenylamino)-2-(methylthio)pyrimidine-5-carboxamide and 34.7 kg 3-chloroperbenzoic acid, and using 21.2 kg (1S,2R)-1-Boc-1,2-diaminocyclohexamine in the displacement reaction. The overall molar yield was found to be 103.3%. The molar yield was above the range of expected yield and was likely due to residual solvent.

### Synthesis Example 4

### Preparation of 4-(3-(2H-1,2,3-triazol-2-yl)phenylamino)-2-((1R,2S)-2-aminocyclohexylamino)pyrimidine-5-carboxamide acetic acid salt

The title compound was prepared in 81.3% yield according to Process Example 4 starting with 42.0 kg of tert-butyl(1S,2R)-2-(4-(3-(2H-1,2,3-triazol-2-yl)phenylamino)-5-carbamoylpyrimidin-2-ylamino)cyclohexylcarbamate and 21.0 kg 99% HCl, 22.7 kg NaOH (50% w/w) in the free base formation step and 7.6 kg acetic acid in the acetate salt formation step.

### Synthesis Example 5

### Further preparation of 4-(3-(2H-1,2,3-triazol-2-yl)phenylamino)-2-((1R,2S)-2-aminocyclohexylamino)pyrimidine-5-carboxamide acetic acid salt

The isolated acetate salt (29.1 kg) was further purified by reaction with free base using 5NNaOH followed by the addition of acetic acid according to Process Example 5 to form the title compound in 85.1% yield.

### Synthesis Example 6

### Preparation of tert-Butyl-(1S,2R)-2-amino cyclohexylcarbamate

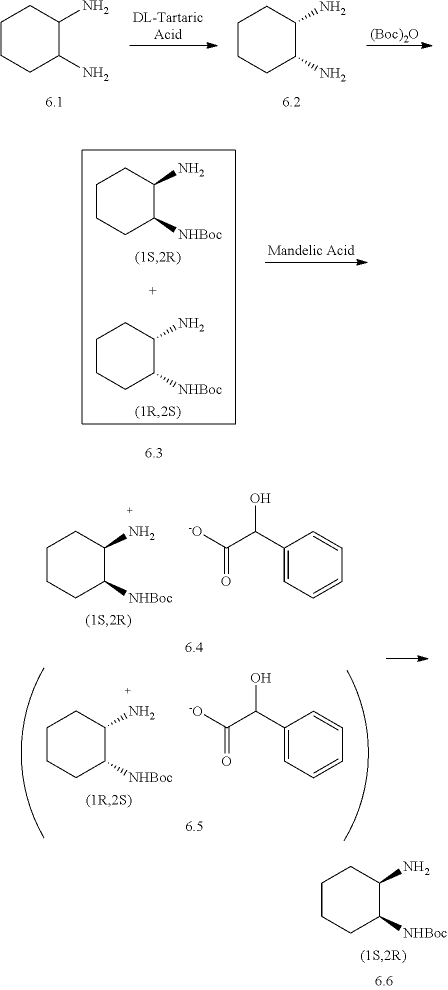

### Step 1:

To a 3000 L reactor was added DL-tartaric acid (247 kg, 1646 mol) in EtOH. The solution was heated at reflux. To the solution was drop-wise added 1,2-cyclohexanediamine 6.1 (249 kg, 2181 mol, cis/trans=36/64). The white suspension formed during the addition. The reaction mixture was stirred for overnight at ambient temperature. Sampling the reaction mixture, it was filtered and then the filtrate was monitored by gas chromatography. The filtrate contained 99% of cis-1,2-cyclohexanediamine 6.2. The resulting white suspension was filtered. 2176 kg of cis-1,2-cyclohexanediamine 6.2 in EtOH (2.24 wt %, 252 mol) was obtained. The yield of cis-1,2-cyclohexanediamine 6.2 was 19.2% from 1,2-cyclohexanediamine 6.1.

### Step 2:

To a 5000 L reactor was added the solution of cis-1,2-cyclohexanediamine 6.2 (2176 kg, 2.14 wt %, 408 mol) in EtOH. Under nitrogen atmosphere, the solution was cooled to 0-5° C. and then HCl in EtOH (3.2N, 107.9 kg, 408 mol) was added. The mixture was stirred for 2 hrs at 0-5° C. The pH of the mixture was 7.0-7.1. A solution of di-tert-butyl dicarbonate (89.0 kg, 408 mol) in EtOH was added slowly. The reaction mixture was stirred for overnight at ambient temperature. The reaction was monitored by GC The reaction mixture contained 71% of the desired product 6.3, 5% of raw material 6.2, and 18% of di-Boc product. To the reaction mixture was added water and then stirred for 1 h. The mixture was concentrated at 30-40° C. under reduced pressure and 2890 L of solvent was removed. To the residue was added ethyl acetate (300 L) and water 360 L. The aqueous layer was separated and then the pH of the aqueous layer was adjusted to 12 with NaOH aq. The oil phase was separated and the aqueous layer was extracted with ethyl acetate. The combined organic extracts were washed with sat. aq. NaCl for 2 times. The organic layer was dried by Na₂SO₄ and then inorganic residue was removed by filtration. A solution of racemic N-Boc-cis-1,2-cyclohexanediamine 6.3, (317.6 kg, 16.48 wt %, 244 mol) in ethyl acetate was obtained. The yield of N-Boc-cis-1,2-cyclohexanediamine, racemate 6.3 was 59.8% from cis-1,2-cyclohexanediamine 6.2. The purity of 6.3 was 96% determined by GC analysis.

### Step 3:

To a 1000 L reactor was added racemate 6.3 (131.3 kg, 613 mol) in EtOAc and D-mandelic acid (0.5 eq). The reaction mixture was stirred for overnight and then resulting solid was filtered and washed with EtOAc. 96% e.e. of the salt was obtained with 34% yield. The mother liquor was reacted with K₂CO₃ (aq.) and free-diamine was prepared. The free-diamine isolated was reacted with L-mandelic acid. 98% e.e. of the opposite salt 6.5 was obtained with 39% yield. The same operation was repeated four times. 89.8 kg of (1S,2R)-salt 6.4 were obtained with high e.e. This salt was recrystallized from isopropanol. 77.7 kg of (1S,2R)-salt 6.4 with 98% e.e. was obtained in 69% yield.

### Step 4:

To the 1000 L reactor was added (1S,2R)-salt 6.4, (77.7 kg, 212 mol) and ethyl acetate. And then K₂CO₃ aq. (35.1 kg, 254 mol) was added. The mixture was stirred for 3 hrs at 20-30° C. The organic layer was separated and the aqueous layer was extracted with ethyl acetate. The combined organic extracts were washed with sat. NaCl aq. The organic layer was dried by Na₂SO₄ and then inorganic residue was removed by filtration. The filtrate was concentrated at approximately 40° C. under reduced pressure. The crude product (42.1 kg) was purified by distillation and the desired product (1S,2R)-N-Boc-1,2-cyclohexanediamine 6.6 (35.4 kg, 147-150° C. 1-2 mm Hg) was collected. Its purity was 99.5%, 98.0% e.e. by GC analysis.

### Synthesis Example 7

### Preparation of tert-Butyl-(1S,2R)-2-amino cyclohexylcarbamate

Step 1. Preparation of racemic 2-(benzylamino)cyclohexanol

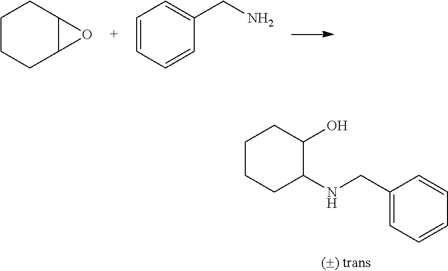

Cyclohexene oxide (14 kg), benzylamine (15.6 L) and water (3.78 L) were charged to a 50 L vessel and the mixture was heated to reflux. When the internal temperature reached 90° C. an exotherm developed up to 97° C. Heating was suspended until this had subsided (˜1 h), then the mixture was refluxed gently for a total of 4 h before being cooled to 20° C. overnight. The mixture set solid during this stir-out. TLC indicated the complete consumption of cyclohexene oxide. The mixture was taken up in MTBE (15 L) whilst heating to 45° C. When the mixture had dissolved the batch was split into two portions which were worked up separately as follows.

An additional 5 L of MTBE was added and the solution was washed with 5 M NaOH (13 L). The layers were separated and the aqueous layer was extracted with MTBE (2×6 L). The organic layers were combined, washed with H₂O (6 L) and saturated brine (6 L), dried over Na₂SO₄, filtered and evaporated to leave a dense off-white slurry that solidified on standing. The solid was recrystallised from heptane (31.7 L) and dried in vacuo at 20° C. The reaction produced a total of 25805.7 g (88%) of the alcohol with a purity of >95% was measured by ¹H NMR.

Step 2. Preparation of D-mandelic acid salt of (1S,2S) 2-(benzylamino)cyclohexanol

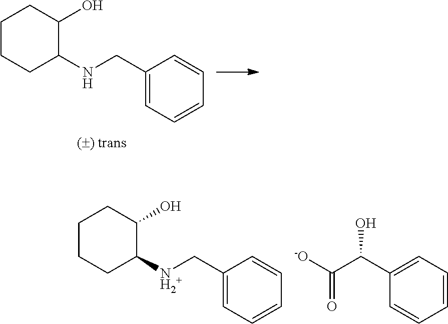

The resolution of racemic 2-(benzylamino)cyclohexanol was carried out in 8 batches and a total of 14479.9 g was isolated. The following yields, purities and % chiral ee results were obtained for each batch:

| Batch | Yield | Purity |
|---|---|---|
| 1 | 2322.8 g (34.7%) | ¹H NMR >95%, 99.4% ee |
| 2 | 2331.4 g (34.8%) | ¹H NMR >95%, 99.4% ee |
| 3 | 2363.3 g (35.3%) | ¹H NMR >95%, 99.1% ee |
| 4 | 2272.3 g (33.9%) | ¹H NMR >95%, 99.1% ee |
| 5 | 2253.9 g (33.6%) | ¹H NMR >95%, 99.2% ee |
| 6 | 2310.4 g (34.5%) | ¹H NMR >95%, 99.2% ee |
| 7 | 311.2 g (32.2%) | ¹H NMR >95%, 98.9% ee |
| 8 | 314.6 g (32.8%) | ¹H NMR >95%, 99.1% ee |

2-(Benzylamino)cyclohexanol (3850 g) and EtOAc (38.5 L) were charged to a 50 L vessel and the mixture was stirred at 20° C. until the solid had dissolved. (R)-Mandelic acid (1430 g) was charged to the vessel, forming a white precipitate. EtOH (3850 mL) was added and the mixture was heated to 65° C., at which point the solid had dissolved. The mixture was cooled to 15° C. over 16 h and the resulting white solid was filtered, washed with EtOAc (13.75 L), MTBE (13.75 L) then dried in vacuo at 40° C.

Step 3. Preparation of (1S,2S) 2-(benzylamino)cyclohexanol

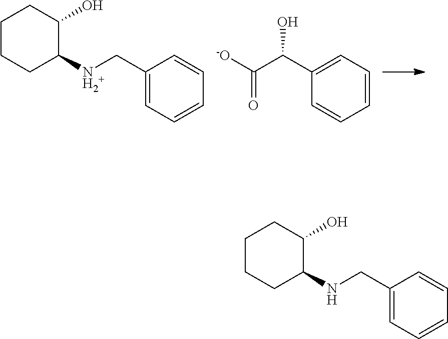

The salt was converted into its free base in 4 batches and a total of 8202.6 g (98.6%) was isolated. The following yields, purities and % chiral ee results were obtained for each batch:

| Batch | Yield | Purity |
|---|---|---|
| 1 | 2054.8 g (97.1%) | ¹H NMR >95%, 99.7% ee |
| 2 | 2089.7 g (98.7%) | ¹H NMR >95%, 99.8% ee |
| 3 | 1872.4 g (97.5%) | ¹H NMR >95%, 99.8% ee |
| 4 | 2185.7 g (101.7%) | ¹H NMR >95%, 99.9% ee |

The mandelic acid salt (3685 g) and MTBE (tert-butyl methyl ether, 25.8 L) were charged to a 50 L vessel. 5 M NaOH (11 L) was added over 20 minutes and the mixture was stirred until a clear, biphasic mixture was observed. The layers were separated and the aqueous was extracted with MTBE (2×8 L). The organic layers were combined, washed with H₂O (7 L), dried over Na₂SO₄, filtered and evaporated. The solid was dried in vacuo at 40° C.

Step 4. Preparation of (1S,2S) tert-butyl(1S,2S)-2-hydroxycyclohexylcarbamate

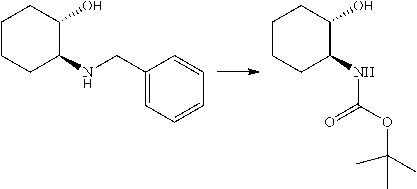

Deprotection was carried out in 4 batches and a total of 8262.0 g (96.1%) was isolated. The following yields and purities were obtained for each batch:

| Batch | Yield | Purity |
|---|---|---|
| 1 | 1987.9 g (92.6%) | ¹H NMR >95% |
| 2 | 2098.5 g (97.8%) | ¹H NMR >95% |
| 3 | 2079.8 g (96.7%) | ¹H NMR >95% |
| 4 | 2095.8 g (97.9%) | ¹H NMR >95% |

20% Palladium hydroxide (201.7 g) was charged to a pressure vessel as a slurry in MeOH (2 L). Stage 3 (2017 g) was then charged as a solution in MeOH (13 L). The mixture was stirred under H₂ (40 psi) at room temperature until TLC (thin layer chromatography) analysis indicated complete consumption of the starting material. The mixture was filtered through an in-line filter and the vessel was washed with MeOH (6 L). The filtrates were transferred to a 50 L vessel and cooled to 0° C. Triethylamine (1371 mL) was added, followed by di-t-butyl dicarbonate (2256 mL), keeping the internal temperature <15° C. When the addition was complete the mixture was stirred at room temperature until TLC indicated complete consumption of the amine intermediate. The reaction mixture was evaporated and the resulting off-white solid was dissolved in DCM (16 L). The solution was washed with H₂O (2×8 L) and brine (8 L), dried over anhydrous Na₂SO₄, filtered, evaporated and dried in a vacuum oven at 40° C.

Step 5. Preparation of (1S,2S)-2-(tert-butoxycarbonylamino)cyclohexyl methanesulfonate

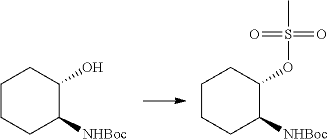 [SMILES: CC(C)(C)OC(=O)N[C@H]1CCCC[C@@H]1O ; CC(C)(C)OC(=O)N[C@H]1CCCC[C@@H]1OS(C)(=O)=O]

The synthesis of the mesylate was carried out in 3 batches and a total of 10890.6 g was isolated. The following yields and purities were obtained for each batch:

| Batch | Yield | Purity |
|---|---|---|
| 1 | 3470.3 g (102.7%) | ¹H NMR >95% |
| 2 | 3493.9 g (103.2%) | ¹H NMR >95% |
| 3 | 3926.4 g (103.6%) | ¹H NMR >95% |

(1S,2S) tert-Butyl (1S,2S)-2-hydroxycyclohexylcarbamate (2480 g) was dissolved in DCM (24.8 L) and the solution was cooled to <5° C. Triethylamine (4022 mL) was added over 10 min, followed by methanesulfonyl chloride (1343 mL) over 1 h. When the addition was complete, the mixture was stirred at room temperature for 1 h. TLC analysis indicated complete consumption of the starting material. Water (9073 mL) was charged to the mixture, which was then stirred at room temperature for 10 min. The layers were separated and the organic layer was washed with water (9073 mL), dried over MgSO₄, filtered and evaporated. The resulting solid was azeotroped with EtOAc (3000 mL) and heptane (3000 mL) then dried in a vacuum oven at 40° C.

Step 6. Preparation of tert-butyl(1S,2R)-2-azidocyclohexylcarbamate

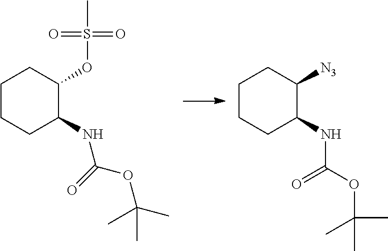 [SMILES: CC(C)(C)OC(=O)N[C@H]1CCCC[C@@H]1OS(C)(=O)=O ; CC(C)(C)OC(=O)N[C@H]1CCCC[C@H]1N=[N+]=[N-]]

The synthesis of the azide was carried out in 3 batches and a total of 3682.5 g was isolated. The following yields and purities were obtained for each batch:

| Batch | Yield | Purity |
|---|---|---|
| 1 | 986.0 g | ¹H NMR >95% |
| 2 | 1189.5 g | ¹H NMR >95% |
| 3 | 1507.0 g | ¹H NMR >95% |

To a 50 L vessel was charged (1S,2S)-2-(tert-butoxycarbonylamino)cyclohexyl methanesulfonate (3455.0 g), DMF (34.5 L), 15-crown-5 (230 mL) and sodium azide (1145.0 g). The reaction mixture was heated to 100° C. and stirred at this temperature for 6 h, before cooling to room temperature over 12 h. The DMF was removed in vacuo at 50° C. and the residue was partitioned between ethyl acetate (17 L) and water (17 L). The layers were separated and the aqueous was extracted with ethyl acetate (2'17 L). The organic layers were combined and washed with water (17 L), dried over MgSO₄ filtered and evaporated. The residue was dissolved in heptane (2 L) and loaded onto a plate filter containing silica (15 kg). The product was eluted using 2.5% ethyl acetate/heptane (ninhydrin to visualize) to give clean product by TLC. The column fractions were concentrated to give a white solid which was dried in vacuo at 40° C.

Step 7. Preparation of tert-butyl-(1S,2R)-2-aminocyclohexylcarbamate

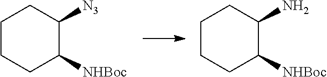 [SMILES: CC(C)(C)OC(=O)N[C@H]1CCCC[C@H]1N=[N+]=[N-] ; CC(C)(C)OC(=O)N[C@H]1CCCC[C@H]1N]

The synthesis of the amine was carried out in 3 batches and a total of 2170.5 g was isolated. The following yields, purities and GC results were obtained for each batch:

| Batch | Yield | Purity |
|---|---|---|
| 1 | 773.2 g | ¹H NMR >95%, 97.8% cis 0.3% trans |
| 2 | 738.2 g | ¹H NMR >95%, 98.0% cis 0.6% trans |
| 3 | 659.1 g | ¹H NMR >95%, 97.6% cis 0.65% trans |

To a 20 L buchi hydrogenator was charged 10% Pd/C (325.5 g) as a slurry in methanol (3900 mL). A solution of the azide (1390.0 g) in methanol (8000 mL) was charged and the flask rinsed with methanol (2000 mL). The stirrer was set to >1000 rpm and the reaction mixture was stirred under H₂ at 1 bar overnight. TLC (EtOAc:heptane, 1:1) showed the reaction to be complete. The catalyst was removed through the pressurized in-line filter and washed with methanol (2000 mL). The solvents were evaporated at 40° C. and the residue was dissolved in 15% acetic acid (5000 mL) and charged to the 50 L vessel. The aqueous was washed with ethyl acetate (5000 mL) and dichloromethane (5000 mL). The pH was adjusted to 10 by the addition of solid K₂CO₃ (1619.2 g). The product was extracted into ethyl acetate (3×5000 mL). The organic layers were combined, dried over MgSO₄, filtered and evaporated to give colorless viscous oil. A total of 1958.2 g of chiral diamine was synthesized with 97.42% cis and 0.68% trans isomer.

### Synthesis Example 8

### Preparation of 3-(2H-1,2,3-triazol-2-yl)aniline

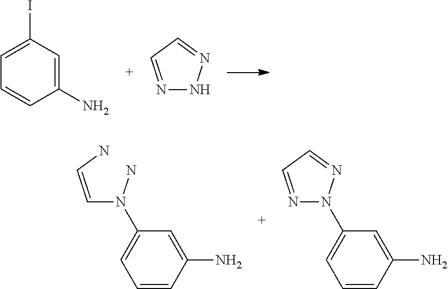 [SMILES: Nc1cccc(I)c1 ; c1cn[nH]n1 ; N/C=C\N(N)c1cccc(N)c1 ; Nc1cccc(-n2nccn2)c1]

The mixture of 3-iodoaniline (3.70 g, 16.9 mmol), 1,2,3-triazole (3.91 mL, 67.6 mmol), K₃PO₄ (7.17 g, 33.8 mmol), fine powder CuI (1.61 g, 8.45 mmol), ethylenediamine (0.60 mL, 8.45 mmol) in 30 mL dioxane and 15 mL DMSO were refluxed for three days to yield as the major product 3-(2H-1,2,3-triazol-2-yl)aniline and as the minor product 3-(1H-1,2,3-triazol-1-yl)aniline in ratio of about 3:1. The mixture was diluted with 400 mL EtOAc, vigorously stirred, filtered through celite, washed with brine twice, concentrated in vacuo, and subjected to flash column to isolate 3-(2H-1,2,3-triazol-2-yl)aniline (1.86 g, 68% yield).

### Synthesis Example 9

### Preparation of 2-(3-nitrophenyl)-2H-1,2,3-triazole

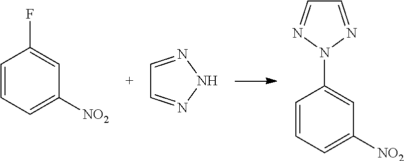

1-Fluoro-3-nitrobenzene (500 g, 3.55 mol) and 1H-1,2,3-triazole (489 g, 7.10 mol) were mixed together in anhydrous NMP (5 L) under N₂ at RT. Cesium carbonate (2.313 kg, 7.10 mol) was added and the resulting mixture heated at 120° C. for 18 hrs. LC analysis showed no starting material. The reaction mixture split into 2 equal portions for work-up with each portion being treated in the following manner: a) reaction mixture quenched into brine (10 L) and extracted with EtOAc (3×6 L); and b) EtOAc extracts were washed with water (2×8 L). The quench resulted in an emulsion after first wash, therefore organic layers were filtered before continuing the washes. The combined organic extracts (˜30 L) were dried over MgSO₄ (1.3 kg), filtered and concentrated in vacuo. The crude material (1.1 kg) was purified by column chromatography (10 kg SiO₂) eluting with 10-40% EtOAc in heptane to yield the desired product in 174.7 g, 26%. ¹H NMR>95% purity.

### Synthesis Example 10

### Preparation of 3-(2H-1,2,3-triazol-2-yl)aniline

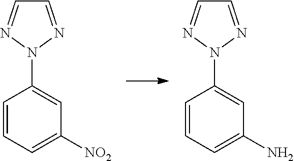 [SMILES: O=[N+]([O-])c1cccc(-n2nccn2)c1 ; Nc1cccc(-n2nccn2)c1]

2-(3-Nitrophenyl)-2H-1,2,3-triazole (194.5 g, 1.024 mol) in EtOAc (1.95 L) and MeOH (970 mL) added to 10% Pd/C (19.45 g) at RT. The resulting suspension was purged with H₂ for 3 hrs then left under a blanket of H₂ overnight. LC showed no starting material. Reaction mixture purged with N₂ then filtered through celite (30 g) washing the filter cake (EtOAc (1 L), MeOH (1 L) then EtOAc (1 L). The filtrate was concentrated in vacuo to yield 3-(2H-1,2,3-triazol-2-yl)aniline in 166.5 g, quantitative. ¹H NMR showed residual EtOAc (5.7%).

### Synthesis Example 11

### Preparation of 2-(3-nitrophenyl)-2H-1,2,3-triazole

A 100 liter reactor was prepared by drying and under nitrogen with stirring. 43.2 L dry DMF was added and then 41.5 kg NaH was added portionwise and the temperature was kept below 40° C. 6.57 kg 1,2,3-triazole was carefully added portionwise and hydrogen evolved. Then, 2 kg 1-fluoro-3-nitrobenzene was added portionwise. The temperature was then raised up to 120° C. and stirred for about 16 hours until the reaction was judged complete (when the amount of 1-fluoro-3-nitrobenzene was about <1% by HPLC). 26 L DMF was removed by vacuum distillation. 68 kg water was added and stirred for 1 hour. The reaction mixtures was then filtered and dried to give 19.5 kg as a light brown solid. The solid was added to 90 kg EtOAc and refluxed for 30 min. The reaction mixture was cooled to ambient temperature and filtered. 13.5 kg conc HCl was then added and stirred for 20 min and the layers were separated and the EtOAc layer was washed with 50 L water and 50 L brine. The reaction mixture was concentrated to remove EtOAc. 36 kg MeOH was added and refluxed for 1 hours and cooled to ambient temperature. The mixture was then filtered to give 5.8 kg 2-(3-nitrophenyl)-2H-1,2,3-triazole with purity about >99% by HPLC.

### Synthesis Example 12

### Preparation of 3-(2H-1,2,3-triazol-2-yl)aniline

18 kg of MeOH was charged into a hydrogenator. 0.28 kg Pd—C (60% wet) was added and then 5.5 kg 2-(3-nitrophenyl)-2H-1,2,3-triazole and the reaction mixture was allowed to degas. The reaction mixture was then pressurized under 5 atm hydrogen at 35° C. and stirred for 6-10 hours (with monitoring by HPLC). The reactor was then cooled and filtered and the MeOH solution was cooled to 0° C. and stirred for 1 hour. The reaction mixture was then filtered and dried to give 4.3 kg product without a noticeable amount of the regioisomer.

The present invention provides a number of embodiments. It is apparent that the examples may be altered to provide other embodiments of this invention. Therefore, it will be appreciated that the scope of this invention is to be defined by the appended claims rather than by the specific embodiments, which have been represented by way of example.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety. From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A process for preparing a compound of Formula (Ia): 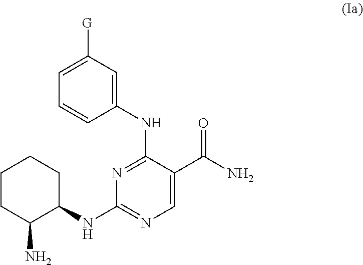
or a tautomer, salt, or hydrate thereof wherein G is heteroaryl;
the process comprising (a) contacting a compound of Formula (Ib) with a compound of Formula (II) to form a compound of Formula (Ic): 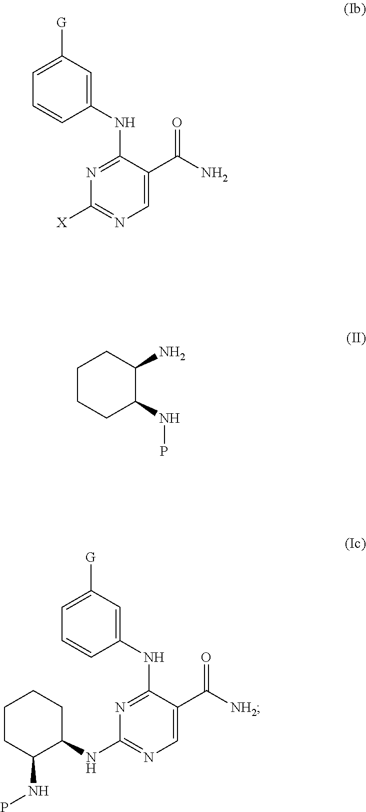
wherein X is a leaving group; and P is a protecting group;
and (b) further converting a compound of Formula (Ic) to a compound of Formula (Ia).

2. The process of claim 1, wherein step (b) further comprises (i) contacting a compound of Formula (Ic) with a deprotecting reagent and subsequently with a base to provide a compound of Formula (Ia) as a free base; and
(ii) optionally contacting the free base of Formula (Ia) with an acid to provide a salt of Formula (Ia).

3. The process of claim 1, further comprising contacting a compound of Formula (Id) with a formamide and an C₁₋₈alkoxide to form a compound of Formula (Ib): 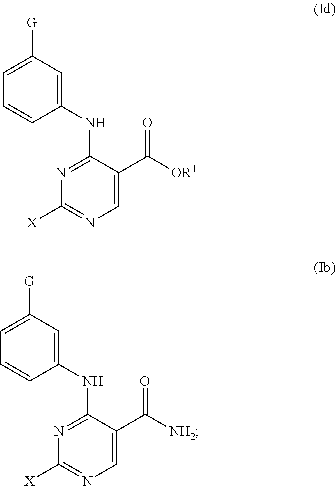
wherein R¹ is is C₁-C₈alkyl.

4. The process of claim 1, wherein X is halo or —S(O)ₙC₁-C₈alkyl; and n is 0, 1 or 2.

5. The process of claim 1, wherein P is t-Boc.

6. The process of claim 3, wherein the alkoxide is sodium ethoxide.

7. The process of claim 1, wherein in step (a) X is —SCH₃ which is contacted with an oxidizing agent before the compound of Formula (Ib) is contacted with a compound of Formula (II).

8. The process of claim 7, wherein in step (a) the oxidizing agent is 3-chloroperbenzoic acid.

9. The process of claim 2, wherein in step (b) the deprotecting reagent is hydrochloric acid.

10. The process of claim 2, wherein in step (b) the acid is acetic acid and the compound of Formula (Ia) is an acetate salt.

11. The process of claim 2, wherein the compound of Formula (II) is prepared by:
(a) contacting racemic mixture (IV) with D-mandelic acid 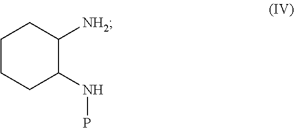
(b) isolating D-mandelic acid salt (V): 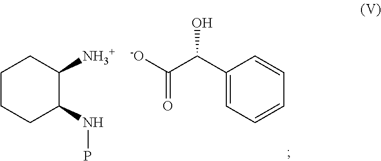
and
(c) contacting the D-mandelic acid salt (V) with base to provide the compound of Formula (II).

12. The process of claim 11, wherein the compound of Formula (II) has an enantiomeric excess of at least 98% e.e.

13. The process of claim 11, wherein the base is potassium carbonate.

14. The process of claim 11, wherein the compound of Formula (IV) is prepared by
(a) contacting 1,2-cyclohexanediamine with DL-tartaric acid to provide a tartaric acid salt of trans-1,2-cyclohexanediamine; and
(b) removing cis-1,2-cyclohexanediamine from the tartaric acid salt of trans-1,2-cyclohexanediamine and isolating cis-1,2-cyclohexanediamine (III): 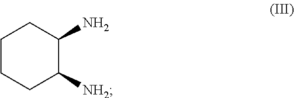
and
(c) contacting cis-1,2-cyclohexanediamine (III) with an acid and di-tert-butyl dicarbonate to provide a racemic mixture of the compound of Formula (IV)

15. The process of claim 2, wherein the compound of Formula (II) is prepared by:
(a) contacting cyclohexene oxide with benzylamine to provide a racemic mixture of a compound of Formula (VI) wherein R¹ is —CH₂Ph: 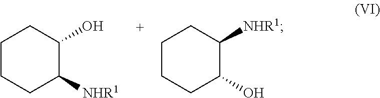
(b) contacting the racemic mixture (VI) with D-mandelic acid;
(c) isolating D-mandelic acid salt (VII) wherein R¹ is —CH₂Ph: 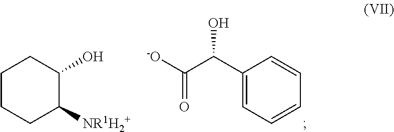
(d) contacting salt (VII) with base to provide a compound of Formula (VIII) wherein Y is OH and R¹ is —CH₂Ph: 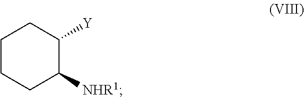
(e) contacting a compound of Formula (VIII) wherein Y is OH and R¹ is —CH₂Ph with a reducing agent and then with di-tert-butyl dicarbonate to provide a compound of Formula (VIII) wherein Y is OH and R¹ is —C(O)OC(CH₃)₃;
(f) contacting a compound of Formula (VIII) wherein Y is OH and R¹ is —C(O)OC(CH₃)₃ with an alkylsulfonylhalide to form a compound of Formula (VIII) wherein Y is —OS(O)₂alkyl and R¹ is —C(O)OC(CH₃)₃;
(g) contacting a compound of Formula (VIII) wherein Y is —OS(O)₂C₁₋₈alkyl and R¹ is —C(O)OC(CH₃)₃ with MN₃ where M is selected from the group consisting of Li, K, or Na to form a compound of Formula (IX) wherein R¹ is —C(O)OC(CH₃)₃: 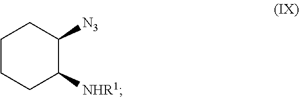
and
(h) contacting a compound of Formula (IX) wherein R¹ is —C(O)OC(CH₃)₃ with a reducing agent to provide the compound of Formula (II).

16. The process of claim 15, wherein in step (d) the base is sodium hydroxide.

17. The process of claim 15, wherein in step (e) the reducing agent is H₂ and Pd(OH)₂.

18. The process of claim 15, wherein in step (f) the alkylsulfonylhalide is methanesulfonyl chloride and Y is —OS(O)₂CH₃ in Formula (VIII).

19. The process of claim 15, wherein in step (g) a crown ether such as 15-crown-5 is added.

20. The process of claim 15, wherein in step (h) the reducing agent is H₂ and Pd on C.

21. The process of claim 15, wherein the compound of Formula (II) has an enantiomeric excess of at least 98% e.e.

22. The process of claim 1 wherein the compound of Formula (Ib) is prepared by contacting a compound of Formula (Ie) with a compound of Formula (If) or a salt thereof and a base: 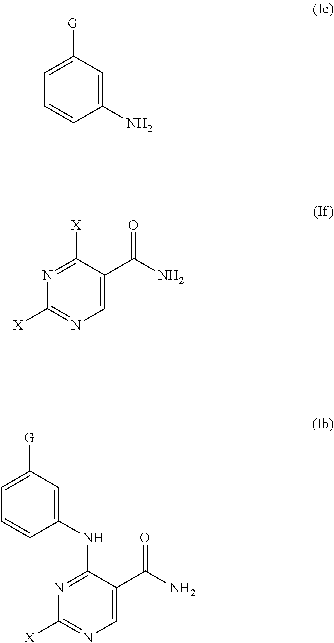
wherein each X is independently, a leaving group.

23. The process of claim 3 wherein the compound of Formula (Id) is prepared by contacting a compound of Formula (Ie) with a compound of Formula (If) or a salt thereof and a base: 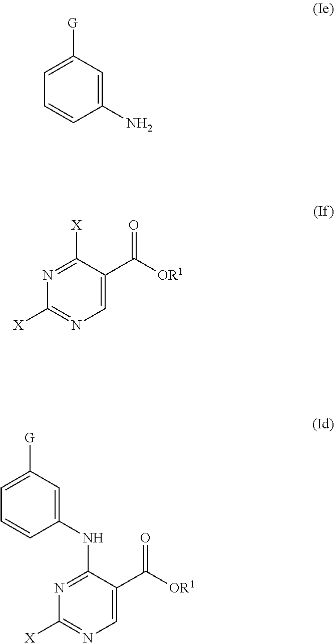
wherein each X is independently, a leaving group and R¹ is C₁-C₈alkyl.

24. The process of claim 22 or 23, wherein the base is triethylamine

25. The process of any one of claims 1 to 24, wherein the compound of Formula (Ia) is

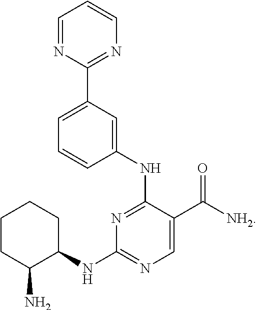 [SMILES: NC(=O)c1cnc(N[C@@H]2CCCC[C@@H]2N)nc1Nc1cccc(-c2ncccn2)c1]

26. The process of any one of claims 1 to 24, wherein the compound of Formula (Ia) is

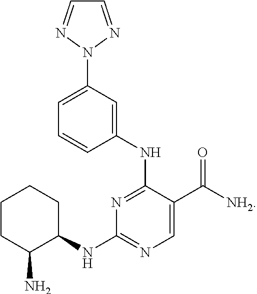 [SMILES: NC(=O)c1cnc(N[C@@H]2CCCC[C@@H]2N)nc1Nc1cccc(-n2nccn2)c1]

27. The process of claim 26, wherein the compound of Formula (Ie) is a compound of Formula (X): 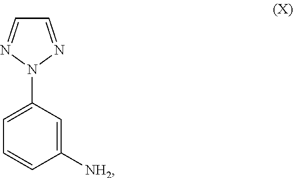 [SMILES: Nc1cccc(-n2nccn2)c1]
wherein said compound is prepared by
(a) contacting a compound of Formula (XI) wherein W is halo with 1H-1,2,3-trizole to provide a compound of Formula (XII) 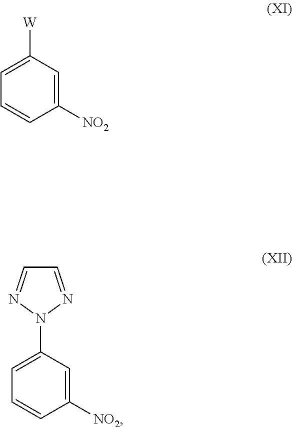
and
(b) contacting a compound of Formula (XII) with a reducing agent to form a compound of Formula (X).

28. The process of claim 27, wherein W is fluoro and the reducing agent is H₂ and Pd on carbon.

29. A process for preparing a compound of Formula (II): 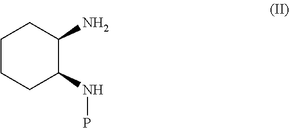
wherein P is a protecting group, the process comprising:
(a) contacting racemic mixture (IV) with D-mandelic acid 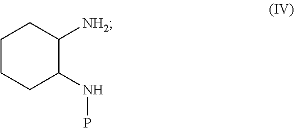
(b) isolating D-mandelic acid salt (V): 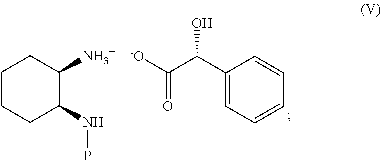
and
(c) contacting the D-mandelic acid salt (V) with base to provide the compound of Formula (II).

30. The process of claim 29, wherein the compound of Formula (II) has an enantiomeric excess of at least 98% e.e.

31. The process of claim 29, wherein the base is potassium carbonate.

32. The process of claim 29, wherein the compound of Formula (IV) is prepared by
(a) contacting 1,2-cyclohexanediamine with DL-tartaric acid to provide a tartaric acid salt of trans-1,2-cyclohexanediamine; and
(b) removing cis-1,2-cyclohexanediamine from the tartaric acid salt of trans-1,2-cyclohexanediamine and isolating cis-1,2-cyclohexanediamine (III): 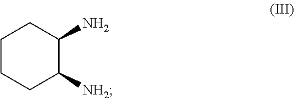
and
(c) contacting cis-1,2-cyclohexanediamine (III) with an acid and di-tert-butyl dicarbonate to provide a racemic mixture of tert-butyl-(1S,2R)-2-aminocyclohexylcarbamate (IV).

33. A process for preparing a compound of Formula (IIa): 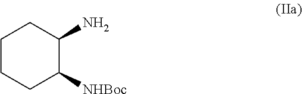 [SMILES: CC(C)(C)OC(=O)N[C@H]1CCCC[C@H]1N]
wherein Boc is —C(O)OC(CH₃)₃, the process comprising:
(a) contacting cyclohexene oxide with benzylamine to provide a racemic mixture of a compound of Formula (VI) wherein R¹ is —CH₂Ph: 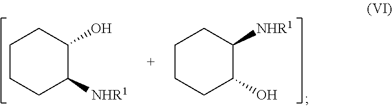
(b) contacting the racemic mixture (VI) with D-mandelic acid;
(c) isolating D-mandelic acid salt (VII) wherein R¹ is —CH₂Ph: 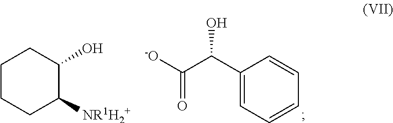
(d) contacting salt (VII) with base to provide a compound of Formula (VIII) wherein Y is OH and R¹ is —CH₂Ph: 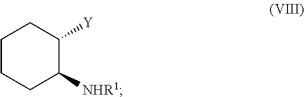
(e) contacting a compound of Formula (VIII) wherein Y is OH and R¹ is —CH₂Ph with a reducing agent and then with di-tert-butyl dicarbonate to provide a compound of Formula (VIII) wherein Y is OH and R¹ is —C(O)OC(CH₃)₃;
(f) contacting a compound of Formula (VIII) wherein Y is OH and R¹ is —C(O)OC(CH₃)₃ with an alkylsulfonylhalide to form a compound of Formula (VIII) wherein Y is —OS(O)₂alkyl and R¹ is —C(O)OC(CH₃)₃;
(g) contacting a compound of Formula (VIII) wherein Y is —OS(O)₂C₁₋₈alkyl and R¹ is —C(O)OC(CH₃)₃ with MN₃ where M is selected from the group consisting of Li, K, or Na to form a compound of Formula (IX) wherein R¹ is —C(O)OC(CH₃)₃: 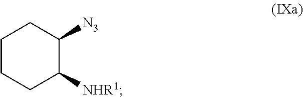
and
(h) contacting a compound of Formula (IXa) wherein R¹ is —C(O)OC(CH₃)₃ with a reducing agent to provide the compound of Formula (IIa).

34. The process of claim 33, wherein in step (d) the base is sodium hydroxide.

35. The process of claim 33, wherein in step (e) the reducing agent is H₂ and Pd(OH)₂.

36. The process of claim 33, wherein in step (f) the alkylsulfonylhalide is methanesulfonyl chloride and Y is —OS(O)₂CH₃ in Formula (VII).

37. The process of claim 33, wherein in step (g) a crown ether such as 15-crown-5 is added.

38. The process of claim 33, wherein in step (h) the reducing agent is H₂ and Pd on C.

39. The process of claim 33, wherein the compound of Formula (IIa) has an enantiomeric excess of at least 98% e.e.
